SEQUENCE LISTING (53 sequences)

SEQ ID NO: 1 - DNA, 1599 bp, Saccharomyces cerevisiae
atgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatag

SEQ ID NO: 2 - DNA, 1026 bp, Escherichia coli
atgaagaagcccgagctgaccgctacctctgttgagaagttcctgattgagaagtttgattccgtttccgacctgatgcagctgtccgagggcgaggagtctcgagccttctcctttgacgtgggcggacgaggttacgttctgcgagtgaactcgtgtgccgacggcttctacaaggatcgatacgtctaccgacactttgcttctgccgctctgcccatccctgaggttctcgacattggcgagttctctgagtccctcacctactgcatctctcgacgagctcagggagtcaccctgcaggacctccctgagactgagctgcctgctgtcctccagcctgttgctgaggccatggacgctatcgctgctgctgatctgtcccagacctcgggtttcggcccctttggacctcagggaattggacagtacaccacttggcgagacttcatctgtgctattgccgatcctcacgtctaccattggcagaccgttatggacgatactgtgtcggcttctgtcgctcaggctctggacgagctgatgctctgggccgaggattgccccgaggttcgacacctggtgcatgctgacttcggttccaacaacgttctcaccgacaacggccgaatcactgccgtgattgactggtccgaggctatgtttggcgactcgcagtacgaggtggccaacatcttcttttggcgaccctggctggcttgtatggagcagcagacccgatacttcgagcgacgacatcctgagctcgctggatcccctcgactgcgagcttacatgctccgaattggtctggaccagctctaccagtcgctggtggatggcaactttgacgatgctgcctgggctcagggacgatgtgacgccatcgtgcgatctggcgctggaaccgtcggacgaactcagattgcccgacgatccgctgctgtctggaccgacggatgcgtggaggtcctggctgattcgggtaaccgacgaccctctactcgacctcgagctaaggagtaa

SEQ ID NO: 3 - DNA, 1047 bp, Rhodosporidium toruloides
atgggccagcaggcgacgcccgaggagctatacacacgctcagagatctccaagatcaagttcgcaccctttggcgtcccgcggtcgcgccggctgcagaccttctccgtctttgcctggacgacggcactgcccatcctactcggcgtcttcttcctcctctgctcgttcccaccgctctggccggctgtcattgcctacctcacctgggtctttttcattgaccaggcgccgattcacggtggacgggcgcagtcttggctgcggaagagtcggatatgggtctggtttgcaggatactatcccgtcagcttgatcaagagcgccgacttgccgcctgaccggaagtacgtctttggctaccacccgcacggcgtcataggcatgggcgccatcgccaacttcgcgaccgacgcaaccggcttctcgacactcttccccggcttgaaccctcacctcctcaccctccaaagcaacttcaagctcccgctctaccgcgagttgctgctcgctctcggcatatgctccgtctcgatgaagagctgtcagaacattctgcgacaaggtcctggctcggctctcactatcgtcgtcggtggcgccgccgagagcttgagtgcgcatcccggaaccgccgatcttacgctcaagcgacgaaaaggcttcatcaaactcgcgatccggcaaggcgccgaccttgtgcccgtcttttcgttcggcgagaacgacatctttggccagctgcgaaacgagcgaggaacgcggctgtacaagttgcagaagcgtttccaaggcgtgtttggcttcaccctccctctcttctacggccggggactcttcaactacaacgtcggattgatgccgtatcgccatccgatcgtctctgtcgtcggtcgaccaatctcggtagagcagaaggaccacccgaccacggcggacctcgaagaagttcaggcgcggtatatcgcagaactcaagcggatctgggaagaatacaaggacgcctacgccaaaagtcgcacgcgggagctcaatattatcgcctga

SEQ ID NO: 4 - DNA, 822 bp, Saccharomyces cerevisiae
gatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 5 - DNA, 427 bp, Arxula adeninivorans
catggctcacttgcggtcaccgcttgcatgaagcgcagattaccacaaaggtcctagtagcttgaagggtgaaaacttgaggtttacaagggcccaaaaactcaattgcagccactaaaatgagcattcaatctataatcagtccatagtcaacaagagcgctcaaaattgatacagtttagtgaatcttgctcgagatgagcgggcgatagttgcttttggggagccctaagtggtacgtgcggcgcgcgggatgtttccctattaggcaaaggccgaccgggtaacccctcgagaaaaaaaaatttttcgccgctaatctggtgttatataaagctccccctgctcttggattttttccttgtcaactcacaccggaaatcgaaggcatttcattctgagtagttctcaaaaaacataatcaaca

SEQ ID NO: 6 - DNA, 910 bp, Arxula adeninivorans
cctggtcgtcctcctcttcctcctggtgtggatcctgcaatacctcagcgatccggttccatgcaattgcctgcgctcgtgtcaactggtactcaggccattgattggtgtagccagaataggtgcgagagaagaacatcaaaatctgctgccagggctctactgcgctccggcggtcgctttcatccatgtacgccatcaatggctcttccggttcattcttatcaacccgcatcgcgtcgtagatgatcggctggaaccctatcgatttaaggtgtagttcggcagacatgaacagcgactccatggcgttccaaattcgctcccgccattccaggtcttggccgctgctgttaacatgactgggcttttcgaccagagccgcaaggtcgatggggttaaaatctgccacgtactttccggccgcccacttctctataaactggttgcgtgccatggcaatccacacgcgtcgtcaagctaatgtcccttccacattactgcggctttgcaatgtgaggttcggtacaattacatcatacgccgcaactacaccagcaaccttaaagaactagtccgaatctgtccagaaaccaattgtcagcaaacaatcagacacacatacatgttttgaccacacaaacaccacaccattatgaccagtcatcattgcgtcctacaaggtcatgttaccatgactgcggtggtattttgtttgccatttgtcataccttcattatgctgcaacgttagacggctgtgtcgatctccgtggtgacaccacaataggccacgtttatccgttgttccgctcattactacaccccttgtgccctgtgtttggtgttgtcatgcctttaattcagtatctgaggccactttaacggaatcacccctgagaatcgcaatc

SEQ ID NO: 7 - DNA, 499 bp, Arxula adeninivorans
cgttccgttcactttcccgtcgcgaccaaattgacttctgttgcctatttttcaactctccgggactggctcgtaagccgcacgcgcttgttatgacagggtcaagctgcccccaacaagctttcaaggcacgccgttatgacgaattggatgacgattatgatcaaaccccgggtcaatcggccgcccgagagaccccttttcgacgcatttgaaaattcaaactcccctagcctagccgacccgcattcggggagtccgcgaaaagttcccggaacagcccatacggtggcctaccgcctcacttgctcggtaatcaccgtataaagccaataaggtgacagagctgttctttgtgactgatagttcggttgatacaagaaggaaagaaaaaaatatcacaatggtgagtagaatttgcgagacgacagtatggcaattgattgtgacgctaacatttccgtaggctcgaaagttctttgtcggaggaaacttcaag

SEQ ID NO: 8 - DNA, 882 bp, Arxula adeninivorans
ttgctgccataccacagtccacctggtacatttctacgctgttccaaggggaataaccgagccgcatgataaccgacccgatcgcaagctcaacaaattgccgtacgggcatacgcgacgagaatttctcggagacctgggagtacggaaacgggtgtcggatttcagtccattggcaactcaaaccgacaattgacaagactcaattgctggaagacaatgccaaaggtataacccacgttacccgcctcactggctaccgggtccgcgatacgaggtccttgtcatgcaccgtggtcagggtccattgtacggtttgaatttgcggttgctcaggcggagccgaacaaaagtcgtggcacgagaataatcgtgcgggggtacacttccccatacctcgtgtatataagtatccatccctactctgtttccatcacctcttgctacggtgaatacacaaaaggtaagtcaattgttgggacctctgtagtatgacgcattaggctaggctgtttttttttcaaacggtttcaccggcatcaccgcagggtcagccttaggggccaccgttgcaaggtactgtttagtgggctcattgtgtgacggttgcagggcaggaattgacccctatctgaggcaaagacgtcattggccccgcaaccaacacaaccagcccctattcacgccattgtcctgattagttttggcacaattgcaattggctcctaatgcagagagaaccctgcaaatgttgcttgattggtcgccccactacagatagtgatgtagtggtgaaccacccaacattggtgtgatatatataaaagcccttgtttgtctattgtgtcattctttcttgaaccaaaaaagactaattccaga

SEQ ID NO: 9 - DNA, 741 bp, Arxula adeninivorans
ccaaacgcgaatccgcccgatgtagaagctccaaacagcggtttggattcagaactagaggtagtagtagtggcaggagtagtggatccagccgaagcgccaaacagtccccccgaaggattgcccgccggcgtcgtcgacccaaaactgaacccgccctgtttaggagtggcagacccgctgttctgtccaaacattgttgcactggtcaaacctgaaggttggtaaacagtagaaatgtgttttcgcacgtgccgtccacatgaacagagtcagatgacagtcagatgacatacggctataaaagcgtcataaatcacctgactaccgcatgactaccacgcgataatcacctgactacaacccgaagattcatcacctcatacccctcggcagatgccgaaagtccgggcaattatcgtgataatcatgacgcccaattgggcaccaattgcgagagcaacaccaaacgacacgcagtgtcaccgcaattgggcctctggtgccactggtgctggcgctgacgttgcctgtcggacaaggaccaccgccctccaatctggccaccagcggcccacgttgatacgttggataagcctttgttgggccgccagccgcgcacccgttgtgttttcagaactgtacctcagggtggtgaggctgcaagggccaagcagtatatataggccccttcggaaccatgggatgtgattagttgaacagacagcagctgattgaata

SEQ ID NO: 10 - DNA, 983 bp, Arxula adeninivorans
gaacacttcaggcacccacacacataccaactcagacacaaacacaaacacagacaaacacaatcgtatgacctgacagcatagtgcattatctatgctcccttcccagctatacacgtcacaatctcagctaatagacctaatagggtgacagttgcactttcccccttgatgtctcatccagaccctcgtcatcccatccgcccaatccttcccagtgcattccaacgccctctcaatggcaaggccaacctctgagccattgacccttaaccagccatagtttactcaactggccgaccgccgtccctttacccattgccatacgcaatattccaatggcctagaggggctgtacggcccattgtccattgtccattgttactgctggtatttttatctcaacgtccccaaaaccgttgtggacagccgcgccgggtgtataggccgccgatcgcagcctctccggaagcggcgcagagcaaaacgagcccagtcgggagtcaaattccgctccttgtatgaattagtccggcacaaagagccccaacggggttaccatgagatgcggaacggcggcaatcagttcagaggctacagtcgtcccctaatcgccatcggatactgccgattgtcgttgtactttacagttttacaagcatagcgataagcccgaagccaaccactcatcaacgagcttaacctgttgggtcgcgtaagcgagcggggggatgctgagtcaggacaacatttgggttggtcagccgcggcccgtagggtagtcgtcgctgaggtcacggctgagtaagcggcaccaattaatccgtttgttacatccgtatgggtggttgcttttttttacctgtacggtttggaacaacaaaaatttggcagggacccattttttttccttttcccttataagcacctcctaggtcccttttagtagcattcgaatttttgacacacacaaaaaaggtacc

SEQ ID NO: 11 - DNA, 930 bp, Arxula adeninivorans
gggtgcggcatttagacagcaacaaagactgcgatcggcgatcagcactaccgttccctgtaaatggtatcaacaaagagcgttccaattcgtcgcttccagcgccggtacccgtaattttctacgtaaaagtgcccccagcactgctgcgcccatgtctaacctaatcataggctcagtgggaaccaccagaccaccctccgactgtgtctgactgtctctgactctctctggccccagaacggctaccgcggagaaagggtaatcggaactttgttctgatgggttgcatgtttgttttgtcccaatggggttagtgcggcaggtacggcaggtgacaggatggcatcgtctcacaagggaacgcagtggaagatgagttttggggggaattagacagagaaatgggcaatttggtggactagggagcagtccatgtgtatctagcagtctccatttagtggcctatgttttttcttatttcttttttgtcaaaaaggagcatttacgtaaccatctacaaaaaaaagaattactaaaatgacacaaaccggggggagccgggatgccgctcacagggtacgcagcgtttgtgcaattcaataaccaccaacaataggagaatatattaacaaagcatacaacagatgtatcccccttggctttgtgcatcgcactgtacctttaatgtttgtgttgacagtcctcagacgcaacccgattgtcccgagtctttgtgatcaaaccgcctcattgtgcatctatttcccattcgggcttgtttgcttatttcccaaaagcaatcccccagggtatataaaggcgcaacgacccgcaccgacggggaactgataaactaagtacagttgttttcaccgttaccggattgattaatcttttttttaactaaaaactactagtacaacaaac

SEQ ID NO: 12 - DNA, 602 bp, Arxula adeninivorans
cgaggcagtaacctcccgttgtcgtcagtaattggggccgaagccgagagaattgacgacggggtgactaccgaatggaggcaggaaacctgttcgtttgtgttccatgtatacgagggcaaaggtcggaccatcgtacacactgagaatgaggaaaagataattgaatgggaaaaggcagatactttctgcgttcccagctgggcaaagtttcggcacattgctgagggaaacgctgacggcccagcttatttgtttagtttttcagacaagccattactagatagcttggccttttaccgagcaaatagcgtatagcaatacattctatatttttttcgagttaaaggtactgataagataagggatccgtcacccattttttgacttgacaccacgactgggagcggagagccgcacaacggttttgtatggggcacagcgaaagggagggagggaaaaaatgaaaaaaatgtgagccgcattagccctaagcagtcacacgcggacccacgattactcctctcccatcgcagcaccatacagagtaaggacgattcaaactgtcaaagtgttcgactgcccaactgaagactcacatc

SEQ ID NO: 13 - DNA, 877 bp, Arxula adeninivorans
tgcgtcggaacgggatatgcattcccctagtttcgccgcagtgcagaatcaggcggtttctttgcaccacaccacatacggaggatgacgggcattattgatgttgaatagtaacctgatcgtgactagtatgacggaacccaacagcaacagccgaccgtttgtgagcgtttttgcggccggtcaggcgagtttttccggcctgccaatggtccttccgtaccctttaccctgtacgctgtacctgccacggataggccgtgctccacctgctcactatggtgggtgcggggaaaacaacaggcaggctcaattgctctgcaaatgggttgagggggtgattgatgtcactggtacaccaacaggggaatgctcggcgttgattttgggccacctcttttgtttgccagagcttgtctctattgtcaaatttaacggtctgcaactgttgcccaaaatgggacaatgatccgatgcctgcatagacaccctgcttgagggtgcgatcgccctaatacgaggcaaaccaagttttccaattgaccttcaattgacgagcggttgttgcgacaggggactggagtgctacctgtttagagttcaaatccgtcacccagcattgaaagtttttccccgcattggatgattgcaatgccgctaacccgctcatccgccaaagttcatagtcccaccctgcctcgacttatcggaccacatggggctcccttatgcgcgcgcatatggcgcttgattgctttttggtcaacgtttgggacaaatttcctttgttaaggcggacccgccagcagatacgaaggtataaatagggctcactttcaccatcttgtccattcaattgcaagactcaaaagtaata

SEQ ID NO: 14 - DNA, 524 bp, Arxula adeninivorans
cccagcccgacttttaacctcaatagctagctacgcaacagacagttaaagctacgtactcaactatatattccattgacaattgacaattacaactgtttcttctcctgcatcgttctcatcctcattggcttatctcctgttatcaattaattataataatatagtagttctgaactaattacgtgatcgcacgcagtacggctgacgcgtattattggaccaacaaaccctaaaaattgtttcatccaattgaacagttcacgcaaccgtgattgtgccaaaaaggcattgccggcctcaagtaggcgcccatgctacgactactgcggtctaggcgctcccgtatccctcaatcgtggcccttttccggtctacccgctgagtcagccccgcccaacaaaaaaagcacaccacaagttcgacatggtccaggggcacggctgcagggttgcggtataaatacagtcaccatttccaccgcacctccgtgctttgtttttcaattggcaacctataacaca

SEQ ID NO: 15 - DNA, 668 bp, Arxula adeninivorans
caggtcgcatgtatgcacggtttttccggcagcaatgctgccgcctcccttgggagtaacatgaacactcacaccaatgtgtggtccaaaaaactgctgacattagttgcaactccggatctttttgccaacggtttgccccggcaccgcccatggggccccagtatacggggttaacttacagtcccactggaagcctttgttcaccgatggcaatgtcgcaccggaccgtgagccgtacatgagacgcacgcaaaactttgtagccgaggcgaaggacaacaattgggttgaatagagccaggggagagctcagggtccctcgggttttgaattatccctgaacctctagcaaaaggttccaaactaagggttgcgcttaactgtacgccttttgatttccggcctgtgaattgttgccccattagggactcaaaccctctaccggcacctcccgaccgagggccgtcctgtgccgaaaaagcaatgtgagattcccttgtggcaggttgggatttgttataatttttttttttatggttggttgaatatataaagaagcagaggcccccaatagaattgtcatcagtcactttttaacactgaactaacaatcatacattaccattaattgatagacattggagaaggaaaagtactaactctaa

SEQ ID NO: 16 - DNA, 1366 bp, Yarrowia lipolytica
tggcagacagtgacgagtcatacattctccgtataatatcgtgtatgtccagacgatagtcgtactcgtactcgttactgtaactactgtgcgagtactcgtgcatgtatcgtaggtattgtatgttcgagtacatacacatacgataccaaacactgcccactgttctgtcatgttagatcatggccaatccacgtgacttgcatgcaggtttggcattgaatattcagcgtggctactacaagtagtacatactgtatcaatacgattgtacatacggtactcaccctttgctacagtatgtacatacaagggcgcacatggcagaataccatgggagaattggcccgcatggagttcagatgagccctaacaacgcccctgttcggcttcagaagcaattggcttttggaaattatttggcgagtgaacaatggcgtgtatggagccgtattcgtgctggtgcttgttgaatcagcccattgcgcgaaattgttggctctcacaactcaacggtctcttttaccctgtcgtgacgagacgctactgtagcgcttgtcggtcggaccacaccaaaactgggcctgtattgcattgtactcagatgtaagcaccaagagctgggatccacgtgatcgcccccacacaagacgcgtccatctgtctattgctcattctccccggcgctctccgatctcttccgacgaaaatgagcacatttcacacgcatctcaagtcagtttggaggcagtagggcgagggtagaggtctggatagggaaaacgagtgtggaaccttattatttggttgggcacatcccaaagacctccacgtttcgaaatcagttgtgttctttttctttgaacttcacgatatttcgtttattcaggtgagtacccacgaaacgcagccaattggttccaattgagtcctagggaggtgacacaaacacacagcgacacagagacggacacacagggctccgtctgtggtgagagatacactagtaaccactactggggcgcccaatgccgtgagcgagagtgtcagcaaagtagtatatggagctatgcacaaatgctaaggcaaattgggatgcacggtgtgtcaatgggataacgcaagttgtgttgctacgccactggtgactctcttgtctggtgatgttgtcttggtgcagtgttggggttgagctcttgtgctgttgggtccgtctgtgtggatgatttgacctatttctgtgtcaagtccacatacaaacaggatctatcaatgccacggaccagtcacaacactgccacgtccgcctctgcgaccttctacctcctcttcgactgcacatgttccctcattctaactaactcag

SEQ ID NO: 17 - DNA, 1000 bp, Yarrowia lipolytica
gggagagcatggagcagaaacggttcgatgcttcaagttcgagtacaagtgcacagtgatgttgcaacacagtcaccactgcctgagtcactctggctgcctatcaggatgtactctcacacatctcacgttcggctcacctcttctttgtcaaggcataaagttcttagaccattgtgactacgcagtttgctccgaaaagatgcatgatcccacccacttgcgcctggaaccggtggacgtgtgctaccagcaggacgtgtatggcacgcgatttattgttgagtaccaaaatagtacttgtagaacgtattccaattcacctttggcttcaccgttgttgtgacccgagctactgtacttcctgtacttccaatctaggcctattcgccacttatagcaaggcaatggtatcaacggtgcgtcgtaactgcggcagtatgcggagagcaggcgtatctatgacgtcggtcggcccgtgggcagtggggaatcaggtcatgtgtttgtttctttgccatattttgcttgtccaatgagctgagtcagaagttcactaagcatcgatctttatggaacaccacgggtactgtagctatggtgacgtaattgttagactacgtagcacaccagactacaagtccatacatccagacagagagtgctaaaaagaaaatatggggcagcatagacatcggaaaccatgggcgatgatagctacccaccacacccaaacagtcagggtaccgtacgtacctgtagtgtgtacttaaccagctcgggtccagtctcgtgccaaacctccgatccactcttcctggtcatctcactttatctggcagtaactctggtccccataccttgctgtcagactctccgcatttaacctgcacaaccctaattcggcctcacacactctccaaatacagataaaacacaaaggtttcgttcaccctatactccgaatcaacgctacctactgcatttctctaccgcaaca

SEQ ID NO: 18 - DNA, 971 bp, Yarrowia lipolytica
gaggtacggcctcgttcaggctatacggaaaagttttcttttgacgtttttttgagtggatttccacgagccatttaggtcatggttggatatcaggtcatggccttggtacgagaagcatcttgtcgactgattagcttggactcatgttatctgcctcggtagcaactccagtgcgaacaagcacactgtactgctgctcacatgcggtttcaaatgcacggggagacgcccagtgccaatgtcgccacatttccaggtcgtcgagttgaccttttccgcacaattgagtccacattgtctacttggtcctggtactacactcgtaccggtaccttttgatccgatggttactttttatgttctattttacattagcgtggaaatagaccatgccatctttggcaccccggaaaaacttgatccaatagagttgttgggtggagctagtgactggcggcaattggagagcttctagaagacgaaccaggagcccgataggaactccgttggcgtgagtcggccccccagtagcaattcgaatcacgtgacgtggagttttccctcgcccgcgttcctggattgtcccggtgtgacgaggccgactggatttgatcacccaaccccacacgacgcataatgtaaatgtatcatcatacagtacatgcccgagtctaatgattggctggttcacggggaccgggagcgtccagtgggccgtatggcggtggttcaaacacctcagatcagctcactatcggctgagacaatccttaactgttggtcggttgccgtttttgcctgctcctaacagctcgcaccgactctaaaaaacctatacgacctggccggcgtaactttgagtgtcgtcaaactctgatatatatatagagagacgtatcccaacagttgatagtcgacaaacgcaaaacagacggacactgaaccccccgcgcttcaaaacaccgaca

SEQ ID NO: 19 - DNA, 1076 bp, Yarrowia lipolytica
gttggatttagttagaaattagttgactggaaaagtcacctgggggttcatttctggtgttacaagaatggaagaacattgagatgtagtttagtagatggagaagacttgagttctaaacaaaagagctgaaatcatatccttcagtagtagtatagtcctgttatcacagcatcaattacccccgtccaagtaagttgattgggatttttgtttacagatacagtaatatacttgactatttctttacaggtgactcagaaagtgcatgttggaaatgagccacagaccaagacaagatatgacaaaattgcactattcgatgcagaattcgacggtgtttccattggtgttatgacattcatctgcattcatacaaaaaagtcttggtagtggtacttttgcgttattacctccgatatctacgcaccccccaacccccctgctacagtaaagagtgtgagtctactgtacatgcttactaaaccacctactgtacagcgaaacccctcagcaaaatcacacaatcagctcattacaacacacccaatgacctcaccacaaattctatacgccttttgacgccattattacagtagcttgcaacgccgttgtcttaggttccatttttagtgctctattacctcacttaacccgtataggcagatcaggccatggcactaagtgtagagctagaggttgatatcgccacgagtgctccatcagggctagggtggggttagaaatacagtccgtgcgcactcaaaaggcgtccgggttagggcatccgataatatcgcctggactcggcgccatattctcgacttctgggcgcgttgtattcatctcctccgcttcccaacacttccacccgtttctccatcccaaccaatagaatagggtaaccttattcgggacactttcgtcatacatagtcagatatacaagcaatgtcactctccttcgtactcgtacatacaacacaactacattcaaaatggtgagtgattgaggagacaattggccggcggtcaattgagcgacacaaaaccccgcgcgcgcacgcactaacacag

SEQ ID NO: 20 - DNA, 997 bp, Yarrowia lipolytica
cccttccgtacctctctgccccttctggacaggtcaatgatagactcagagcgacacacatgtctgacgtaccatgttagaccttgtattgacctggacgaatgtgtgtgaggagtgaggcaggccaagacgaaccacggtctttatatatgcccacggagtgacacggtctgtgtcgtcaccgcagctccactcaccacccgcatcatgatcgtccaaccagaacccactccccagtttcgacccaacaccattctcaactgtaagtatgagtaccacagtgatactcgcccagtgccgcactcgtactgtagccactccactgcaacatccgtatcgtattgcaccgccccgattcacctgcttccttcaagccttcaaccacgtactgctccacctcctaccgttgagcccactcggatcggccagagtcatgtcttagggtttggctgcagttgtggcgtaaactatggagaaggcgacggaaacgagagcgctaccggtagcgacttggcgacacgtctggctcgggaagggggccgttgcagagaccaagacttccgtcacgtgaccgctgtttggtcaattctaacgcagttattttccgtctgattcgctgatacgagtactcgcttgctgtagatgactcagaccaagacaagagaaggggaaataaaaaaaacttccaaaaaaaacttccaaaaaaaaaaaatcaaaatttgacaaaccttttctgcctgggaccaggaactttgtgagtccattgagggagttagccacccatcagccacagccacagtttggacaagaagtaaaagtggatatatttatgttatggagaccatgtagtgttgtgggagggaggggtttttttgtttgttttggctgagtaatcaacagcaagtggcgtatatcgtatatctatcgtgactcagactattcaccgcttgtatggtgctatctcgacttgtgcttagtctcaggtacacgtgattgc

SEQ ID NO: 21 - DNA, 1691 bp, Yarrowia lipolytica
tgcaaccagtctccgtggtgtgcagcatacattgttcccgcctctccttgtcttgttggaaggccgatgtcgctgactgtatgtaccgttttttttgtaccgtagtacatgcagggcttggtattttccaactacagtacatacaggtcttagagtgctgattggagatagatatgaatggagtgtacgagtggaaacaaagcgggttagatatgtgtacttgtacatctgtgatattggtagtattgacaagcggtagtcatttcagtgcatcgccgtgccctttctactatccccttgcgccatcaatctcccccttcatcaatccacctctggcagctcttctagaagacctttttacagtctcccaattttatcgtctagtgacggcagaccttgtaagcagatatgtatcatgagtcacgatagctggacagaccaatggcatgcgggcaaataactcccacagacgctctccctccggcgcacaaagcctcgtgctctgaacacgccccagttgatttgacagctctcaacattcgtgtgaacttttttagcgggaaaaagtaacatgacgttgaccgtgcggggctacatgtagcagctgggtgtgctaactacggatacatgcctacaacccccacaagtcaagaccattgcgacgcggaaacaggagcccgcaaaagaggagaaaaacaacggcgagactcgggggcggagtgggtcacgtgactttcctttttcccctcacctggcccgctccgtccatatctctgtcgtacaagacaatattgtcgcaacgcaaaaggtccataaattactgggtagacgcaactctatttgaaggcaacctaccgtttgcttttagtgttttggttttgttaccatatccaaaaaaaaaccatatatccaaaaattccgctgcaccatctcttcttctctccatcaactacccctgcggagaaattcacaccacagttacaatgatttacaccgccaattcgtccccttccaccaacctgcagtggctcagtaccctgaacacggatgacattcccaccaagaactaccgaaagtcgtccatcattggtactatcggtgagtatacttatccacagaccagacgccgattgcgcggtttggtgcacaattcgacgagcccacaagaggtaggcgtcacaggataacggacccgctcatgtgaacatgtggcgagcccattgtacccgtgtcgcctgcccccaagtcgattcgccgaatgcgctcaaacgctggctcggtctccgcctcaggcctcagtaaaaacggcaaactaacagcaggtcccaacacaaactccgccgagatgatctccaagcttcgacaaggtgagtaaccataatgcgacagcagtgtgcgccgtgacccgattcgcggtggccacgtctatctgtcccttctttcgttaccccaattggcaccgtcgccttattttttggctttggtttcccgggtttgtccaatacacggctcatgcgcatgcacattttttccggtcggataaacccaacgaactctaagtgacaaacatgaaatgaaaacgacgcaagtggtaagggcgctaatggtgacgttcatgacgttgccagtctggtgcccttatcgatgacgtatggacccatgtgtctatcatgccgcaatactaaccacag

SEQ ID NO: 22 - DNA, 996 bp, Yarrowia lipolytica
tccagactacttgccacaaatgcagcgagctgcacattgatgcgttcatgcaagctacaagtacgagtaatttgacgtattgggcacttcaaggcagtctttcgaaatggccaatctgggagctcgctcaccctccgagataactgttgggcacaccagcaggtctcagcaacggttgaaaatgggctctcagttcaactaatgatccaagaaaatacaagtacgatgttgtgattggtcggactacttgtagacgacactagccaaagcgaaaaggcacccaccctatctgaatgctgagctgtgttcagccccaactcggaatgctgaactgttgtaagtcgatagccgatagatatatatcgtagcaaacacaagttgttgactcaaacgcattgacaaggaagtacagatccgagaaattgtgccgtgtcaactgctcccaggcacggtctcaattggggctatatctctgtatagagtaagccaggttggccccccacccacgagaaatgcaccaaccagtcggcgagctcaacagccgtatgggagcctctcgtttgatgtatgtgtgacaggaggtgtatattggggctactgggtgaaaataaaaacgcgagagagaatataggggtttcagcgaaatcccagtggagagaccgaatcatagtattataactatgacagtatcgtgcgctctcctctttcatcacttctctccaatatgcgtaccatctatcaccactctcttgcttagcctccctccctccctctctctctgttagacccccacacgctcaacagtactcaatatccgcgcagaaaaataaggttggtgggacatttctccggtgtgagcgattagtgggttgtggtggtgcccatagcagacctaaaattgactctcctcacttgacaacacacagatagaacctgcaacttcatccaagtggcgcttttctcaatccagtccgtgtgaaaacaagacaattgacc

SEQ ID NO: 23 - DNA, 985 bp, Yarrowia lipolytica
gaccaacctaaattagtccgggtggacgtgtcactagaacgttgtaataccaaggtagttgcgcttgttttgaccaaaaatgtgtgacaaaacattgccagtgtatccagtctgggaattgagtcgttctataaccgtcatttccactccacttccgcgcaacgcgctgctgagcactcggaaaattagctcgaaaagtttttccgggttatgtgacccgccagcaggttaggctctattctgttgggaaataactctcaaccgcccctccgagctaaatctctcactacaagactcttcatcgacaaacgatatctgagatctttttcggtcccacagcaacaagccacaaacatgtctgccatcaagcaattgaaccgtctggccgccaccgccaagacctctgttctcaagcccgcctccaagcagattctgctgcccactgctggccagcaggctgccatccgaatgatgggccagacccgagctgcctcaaccgaaggcggcgccactaacgtgagtattttttgtgtgaacgacacgatatatacacgacggccgtgcgcttgcggcttcgcgatgcgccctgaatggggcaactcgagcgttgtgtaacgggtgttcatcaacagcaaacagtgcttttcggacttaagacatggcagaagaagcaaacacggttatagcgagagagatcacaatggagtgacgagctttcagtgatatttgccaccagtcaaattttcagcaactcctgaaacgcacccattttatcatcattgtgacgcggacattcagcctcatgctgtaactgcactccgtgtctggagctgccggagttatcaaagctgtaggtgccaattgtgaaatagcgattcggcgattcagccgttgattgcgttcctgctatgtcgcaattgtacaatgctctgtacacttccaacaacatcaacatgacaacacccaaaacaacgtactaacctag

SEQ ID NO: 24 - DNA, 999 bp, Yarrowia lipolytica
ggtgagtcgtgtcgctaaaaggtttgcaatgggctcccccaaaggcttttggtggtttgtagggcggtgaaaaatttgtccattttagggccaagatttagacgtgtcgagatggggaggttttggaacacgccgaatcgcatcgacacgactcccctccgcctgaaccacaacctcgccggtcacatgacatggctcctgcacttcggatacggaagcccggatcctttatgctctaccccggagttgtacctgtccaatagaacaagagtcaattggccttactcgcatgcaactcaaacttgggccggggttgagaggtacagttgacaacgtgaaaataagaggggggggaggttaaggcctcaggggcgaatttgagagcacttatatagacaaatccgcaccgaagtgacaacatggacaatgtgacacgtagatacacgccggatccagctgtccacacacatttatcccgaaaaatagcccgcatcacatgcacgtctcgtaaaaaaaaaagagctgcgggccaaaggaccaataagtgccgaggaatgttaagccaaaagaacaacgacgatcgccagacaggtttagtgggagcagcagcagcagaggccgtgcaacggcaggagagagaggtctggcgaaaaggaggagacggggtgttaattgatttgcggattttccgcccagccacaaaaatggcctattttggcgggtttaacggcgtcccctccaattaatccgaaccccgtttaccacgcagcctacactatgtactgttgacaacaccccatgacggtagtctccggagccgagccggacttgtgtttaaaatcggcacgattttgttcagaggttagggttcaccctggctaatagattggcgctgattggcccgaccaaacccaaaatgggcactctgcagtgtttataaaacctctccgaggcccacgattcaactttctcctttccgctctaacaccacatatcaca

SEQ ID NO: 25 - DNA, 999 bp, Yarrowia lipolytica
atggtgcgtggaggctttggcatcctttctacttgtagtggctatagtacttgcagtccaagcaaacatgagtatgtgcttgtatgtactgaaacccgtctacggtaatattttagagtgtggaactatgggatgagtgctcattcgatactatgttgtcacccgatttgccgtttgcgaggtaagacacattcggtggttcaggcggctacttgtatgtagcatccacgttcatgttttgtggatcagattaatggtatggatatgcacggggcgtttccccggtaacgtgtaggcagtccagtgcaacccagacagctgagctctctatagccgtgcgtgtgcggtcatatcacgctacacttagctacagaataaagctcggtagcgccaacagcgttgacaaatagctcaagggcgtggagcacagggtttaggaggttttaatgggcgagaaggcgcgtagatgtagtcttcctcggtcccatcggtaatcacgtgtgtgccgatttgcaagacgaaaagccacgagaataaaccgggagaggggatggaagtccccgaacagcaaccagcccttgccctcgtggacataacctttcacttgccagaactctaagcgtcaccacggtatacaagcgcacgtagaagattgtggaagtcgtgttggagactgttgatttgggcggtggaggggggtatttgagagcaagtttgagatttgtgccattgagggggaggttattgtggccatgcagtcggatttgccgtcacgggaccgcaacatgcttttcattgcagtccttcaactatccatctcacctcccccaatggcttttaactttcgaatgacgaaagcacccccctttgtacagatgactatttgggaccaatccaatagcgcaattgggtttgcatcatgtataaaaggagcaatcccccactagttataaagtcacaagtatctcagtatacccgtctaaccacacatttatcacc

SEQ ID NO: 26 - DNA, 999 bp, Yarrowia lipolytica
tgaccaaccttgtttggtagatgggggggaagcgaaccggcaatattccacaatgtgctggcatttacttgtgctggcaaaagaggcacaaagaatacttgtagtcggagccactcactgtcccacaaatagctccccgctgtcaatctctcctgcaccgcctgctcacatggatgctaagccgcactaggtcgcatatatggctctgcactaaaaattaggggtcaaccacagtgcggtatttttagattcgcaccaagcagcgagtaagcaaaaatacgcctaccggggtccgatattattcaggaggtgccattagaggagggcagatgagagtcggatatcggagatattaccgaggctataattaccccatccacgcctttcacccctcccactctctccctcaccgcacaccaacccaccactttcaaaatataccgcaacattgacataatctccggtacagtggttagcaccgagaggaccccaaaaagcttgggggagatagaggtaggcttttttttgtcagtcaaatcgtatatgccaatacacacacacacacacacacacacacacagtttcgtacataacagtatattggaagggagtgtgcttggcaaagacaggagaagacggtgctgttagagggcaatccagacgggctagagctctgtaactttcggatcgatttcaattcctctagaataccaaataccagtggttaagcggctcatttaccagtcctaataccccctccaccagccaccttcccctattcctcggcagtgcttttttacctttgagatgtggccttgtctccgttacttcccaaccgtgagtgctgtgtggtgtgctggacagtgcgacataactaaccctaacccagacgagccagcgcaccccaattttgtgtttgccaactcctacttttctcctctcctccatcggtatttcatcgacaaatctctttgctaccaacaaccacacaaattaaaa

SEQ ID NO: 27 - DNA, 452 bp, Yarrowia lipolytica
tgtgtgtttggtcgaggtttttttctgttcagtacagaccttgtgtggtgaggaacagcaatagcaagggtggcttttgattgggtgcaggtgcccttaccctgttgggaggtttgtctaggtgcctgggatggaggacaatgttttgtcactgtcaagacgggatattgtggggatttgagaaatatatttgatcagccggtctcgaagattatattcgcgctttcgcctttgaaattgctccttttgttgccgtttcgaactgtagtctcgtgctactgagtctcatgttaatttttgtttcggcctcgacttaattaactctaaccaatgttattttcgtgcattaacgaaactcgaacgcacgatcagtcacactctccaccatcaaatatcacgtacagactggtaccccatacatactaccatctgaagacgacacaccacccatc

SEQ ID NO: 28 - DNA, 1000 bp, Yarrowia lipolytica
ctcgaataaggcactatttaggaccagaccacaccccgcggatgtcaagccgaaccttgttgcataaagataatactagtcaagtggggtgtcgacccgatgagagaataaaccgattgcaacggtttttatttcattcgcttcttccagcagacactcttggttttcttcctcacagctttccgccattatcagctgcgtgtatcgtgagtatattgggagtgagagatgccctcacgataagacaacagctatagtacaaatgttaacacagatgtcagatcaagcgccgccaaactcgcccggaacacgggtaccaggggagatcggtccccaacaatcttcccagcaagttcccatggcttataccatcccaggaacaacaagtacagctctagatgaggagatctcggagtaccgtgacaccaaccgaacgaccaagacccctgggatagacgagctgacacccacagcgttttatgacaatcgtggtgttaggcatgagcataggggaatatctgaagagatgaagaaggagctcaagagacaggagagacgccagcacgagatgttgcaacagaagcagcttgagctgagacaacaggaagccctacaccaacaccaaatgcttatcattgagcagcagaagcaagatcagattattcaacagcagaaacagctgcaacaactgcagcggcagcaacaggaagaggtggtcagacaacagcagctgcagcaacagcagcaactgtaccagtactatcagcaacagcaacagcagcaacagcagtatgccgcacacatgttacaattcgagcaacaaaggcgggagcagatgcgacaacttcagttggcccagtaccaggcatctcaggctgttcagacacatcatcaagatgtttctcatctaaccccgtctgttcccgtacctgcagccgtaacacagcctcctgcctccgtagcacgtacggcatcagtctcagacatgttggtacctcct

SEQ ID NO: 29 - DNA, 1000 bp, Yarrowia lipolytica
gtagatacacggtaagtacatactatatctatagatgatacattttctttttataccgaccgcccaagccacacggcaccttaattaaacggccactttgacatgagaccgagctacaaaccagtcgactacaagtactgtcaaagagtcgaaatttgtggagtcgggagtttataatgtccatccaagaacaccctcatttcctgctcgtcttgtgtttcagtagctaatttcacatgtaaaacggcggtcttgatccaccctgtcttaactccggtcggactttgctgccataacgttcggacacgcaactctttcccaaatccaacttacagcatcttacctaatcacacctgccctcacattaggcaccaacctaaacccaagctcaaccgtcgtcgactcagccccgaagaagcaggtactcgtgcaaatatataacgaacagtttaacggcggcccggaaaaagattcggtcgtcacgtgacctacctccaccctaagccggtcccttcaccccccacttttctcactgttctcacttttctcacccccactgtggctctatcaaactctacgatgacacacaatggcagaaaagtgcctctgcatacacgatccaataaaacggtcagtacacgcaacttagtgagggggaggggttacatccagcaggtggtgctaatgttacggcagcttttcagtagtgtgctcgatatttcagcccccgttggaccgcgaaaagcactctacactcgtcttctagtatgttcggtcgtgtcccacgcacttagttgcgataagcgctaatcatgcttttctttgtctgtgcggtggcgattcggaacataatcactgtaagcggcgcatgttgaaccttattttgcctttgagcccacacatggataacacctcatatataacctgtcccctccgctaactctcttgcttctctacaacataacctgttgaaccacaaaacacctaatcaacaaacaac

SEQ ID NO: 30 - DNA, 989 bp, Yarrowia lipolytica
gtcttagtgggactggaaggagtatcagtctcactggttaactgtactggctagaccccggaaagggatggctgtgtgcttgtggttcattgggtgcggtgtggtgtctacaactcgtgttgccagacctggacaagggcatttgtgaatgtgacggtactcgtaggttcaccagagatggtgtcgaacgacacatgatgagagtggaagctccttggatgccatcgacatcacgtgaacctgtctgatcgtccatcgctggtttgtaggacgcgtttgaaggttccgacttgacgttgttggtatgatgcacgagtacaggcgattgtaaggtggtcgagcgtgttttaatgtacaggtggaagtaattgtacttgtatcagggcctcttgcagctcgtcttgtgttgttcgcatcaaatgacactcgtcttgtacagtacagtctccatgacttgctccagattatgtatccaaaacaggggttgtatacttgcagagtacaagcacaggcatacgtatgtacaagcctctttatatctttaagagtacaagtaaacgtactcgcacttgtacttgcaccggcgagatgtatggtcgcagaaaacctgtcggcagccctccgtcctccacatacgaacatgactgacttgcatctttcacctgttcagcaagtttcatactgcactagtccaaataggtaaatcaccttggcctcctatttgggacagggtaagggcgtccagaagaggacaccagtgaaattacataatacaagctgcagtacttgtccgatacgacctgtctcgaaacagccgtttggagcagcgcatttcttgcccaatcaattccctgactactctcactcttccccaacacggtgctttttccccattctggtcacatgactgacacgctccacctaaccttatctccaaagaccacgacatacgcatctctccttcagaggagtttcggaagtctagccca

SEQ ID NO: 31 - DNA, 940 bp, Yarrowia lipolytica
aaggcgagcgaacggctttgtccagtggtcaattttcaagtcaatttttggctaaaaaaaagaccaaattgcagccatccaaactggtcactactcgaccaatatggccgatatttcaatccacatcgaaccagtaaatcagaatgaaccaccagatcaatgaagaacaacaaaatcaaacgaaaaactccctcgggcccgcatgctcccgccaaatcgacaaaatctcttctcccataggcgacattgaccccatgcaatatcggtgacatttgtaaataagatctgaactttaaattatcatactttggtggtgtatggtgcgtggtccacgtggggtaggggaataaaaaaattggaacaaattgggaaatatataaaaattgaaaaataaatggaaaataaaaaaaacgtggatctttcgatgaataaaaatcaggctaatcccagacaaagatcgggagtctttctccctgagccaacgtcatcctgactaatgaaaacatcaaataaaataaatctgacacctaaactaaccaactttatttgggccaatgagacggctgaaagtccgcacgttgtggggggaaatggacaaagtttattttaaacgtgaaaagttggggggaaaaaacaaaaaaatacgaaaatgtagccctgatcggtcacagcccaattatcccctcgaaaaaaatcccctccaaatccccatttttctaccgccattttcgtccatacttttcgataaccctaaaaaaggtcatctatcagtctaaatcttgtattaacctcgaagactaaccgtaacttagactaatgctaacgttaaaatacaactctaaatattaaccgacatcaaaccccgaaaagaatatataatcgtgaggccatcctgaggattttgtctccatcgaattcgaccaccacaaactcctctaca

SEQ ID NO: 32 - DNA, 709 bp, Yarrowia lipolytica
tggcagacagtgacgagtcatacattctccgtataatatcgtgtatgtccagacgatagtcgtactcgtactcgttactgtaactactgtgcgagtactcgtgcatgtatcgtaggtattgtatgttcgagtacatacacatacgataccaaacactgcccactgttctgtcatgttagatcatggccaatccacgtgacttgcatgcaggtttggcattgaatattcagcgtggctactacaagtagtacatactgtatcaatacgattgtacatacggtactcaccctttgctacagtatgtacatacaagggcgcacatggcagaataccatgggagaattggcccgcatggagttcagatgagccctaacaacgcccctgttcggcttcagaagcaattggcttttggaaattatttggcgagtgaacaatggcgtgtatggagccgtattcgtgctggtgcttgttgaatcagcccattgcgcgaaattgttggctctcacaactcaacggtctcttttaccctgtcgtgacgagacgctactgtagcgcttgtcggtcggaccacaccaaaactgggcctgtattgcattgtactcagatgtaagcaccaagagctgggatccacgtgatcgcccccacacaagacgcgtccatctgtctattgctcattctccccggcgctctccgatctcttccgacgaaa

SEQ ID NO: 33 - DNA, 997 bp, Yarrowia lipolytica
gttggatttagttagaaattagttgactggaaaagtcacctgggggttcatttctggtgttacaagaatggaagaacattgagatgtagtttagtagatggagaagacttgagttctaaacaaaagagctgaaatcatatccttcagtagtagtatagtcctgttatcacagcatcaattacccccgtccaagtaagttgattgggatttttgtttacagatacagtaatatacttgactatttctttacaggtgactcagaaagtgcatgttggaaatgagccacagaccaagacaagatatgacaaaattgcactattcgatgcagaattcgacggtgtttccattggtgttatgacattcatctgcattcatacaaaaaagtcttggtagtggtacttttgcgttattacctccgatatctacgcaccccccaacccccctgctacagtaaagagtgtgagtctactgtacatgcttactaaaccacctactgtacagcgaaacccctcagcaaaatcacacaatcagctcattacaacacacccaatgacctcaccacaaattctatacgccttttgacgccattattacagtagcttgcaacgccgttgtcttaggttccatttttagtgctctattacctcacttaacccgtataggcagatcaggccatggcactaagtgtagagctagaggttgatatcgccacgagtgctccatcagggctagggtggggttagaaatacagtccgtgcgcactcaaaaggcgtccgggttagggcatccgataatatcgcctggactcggcgccatattctcgacttctgggcgcgttgtattcatctcctccgcttcccaacacttccacccgtttctccatcccaaccaatagaatagggtaaccttattcgggacactttcgtcatacatagtcagatatacaagcaatgtcactctccttcgtactcgtacatacaacacaactacattcaaa

SEQ ID NO: 34 - DNA, 983 bp, Yarrowia lipolytica
tgcaaccagtctccgtggtgtgcagcatacattgttcccgcctctccttgtcttgttggaaggccgatgtcgctgactgtatgtaccgttttttttgtaccgtagtacatgcagggcttggtattttccaactacagtacatacaggtcttagagtgctgattggagatagatatgaatggagtgtacgagtggaaacaaagcgggttagatatgtgtacttgtacatctgtgatattggtagtattgacaagcggtagtcatttcagtgcatcgccgtgccctttctactatccccttgcgccatcaatctcccccttcatcaatccacctctggcagctcttctagaagacctttttacagtctcccaattttatcgtctagtgacggcagaccttgtaagcagatatgtatcatgagtcacgatagctggacagaccaatggcatgcgggcaaataactcccacagacgctctccctccggcgcacaaagcctcgtgctctgaacacgccccagttgatttgacagctctcaacattcgtgtgaacttttttagcgggaaaaagtaacatgacgttgaccgtgcggggctacatgtagcagctgggtgtgctaactacggatacatgcctacaacccccacaagtcaagaccattgcgacgcggaaacaggagcccgcaaaagaggagaaaaacaacggcgagactcgggggcggagtgggtcacgtgactttcctttttcccctcacctggcccgctccgtccatatctctgtcgtacaagacaatattgtcgcaacgcaaaaggtccataaattactgggtagacgcaactctatttgaaggcaacctaccgtttgcttttagtgttttggttttgttaccatatccaaaaaaaaaccatatatccaaaaattccgctgcaccatctcttcttctctccatcaactacccctgcggagaaattcacaccacagttaca

SEQ ID NO: 35 - DNA, 1000 bp, Arxula adeninivorans
tgcacctccaggctcagggtccccctgtccactgtcctatccaccatccactgttccaccccctcttagacctcagccagacgccgcagcgggcaagcagcccgggtttacagagcgctgcgggcatcggcatgatgcgacagggcctcgatgagcggggatactggaccagaccacggaataaatccttcggaaaagtgcgctttttgaaattggccgacccggcgaatcaggccaggtcaaatcccgcccccgcttccccacaattgaccgatcctgaacatgcacaatctatgacaatggtccgcatcaaattcgcttgcaatagcacttagcggtcgaggtgtctaaccctgtcgaggtttgtgaccgctaacttcttgcaagagcgaaggatgcaaggcgctccttcctgaataggcaattgagccccatgtcgtgaggcttaaagcgtgcttcttgccgaatccggaaacaacgccgccgatgatatgacaaaagccaacaaaatacccgctggagcgataacgtaaggggttggggtatcaacggacgcggcaaacaagcctgtgaaccctttgcgagccatggtttggccttagtttttgtctcccgctatggttacattggctctcgcatgctatggtacctcatctcatcgaaaatttttcaagaggcgcataatggctgtctcgggcaacggtttgcacacggctacgtcggttctcggcctatgattggctctggctttatctctatccgcccacacatacttcaaaaggaaattgagactatgcaaaaagcaattctgggtgtcggagtgctgtatgacgattccataagattttgccgggtcgtatcgaataaaaacccctcttttccccccattgtcaccagattcctgttgtgtttttttaataatctccttttcaacccgcttgttggtggtttgaaaatatacccattttttctaatttaatttgctctttgttagcgtaaa

SEQ ID NO: 36 - DNA, 1000 bp, Arxula adeninivorans
caacttgtgtagtagacaaagtgtaaaagaaagcaatttgcgactttagcgctgctctggcacgtgtatacccggtcagagtgatgcaattgagtgagcctggcatggagattatgaccgggcccatcggattccgagttttttgatcccggctccaacttcattgctcatcgcaccctactgtattgaactgacgaccaacagggccagtttctccaaccaaaacagtgcagtctaattagtttgtaattggcaactttagccttagtctctctgaagagttctaccccaattccccctggaccaccccagaacccatgttgaccaggatagcgccgcatgcaggggccacgtgaagcagcgcgataagattgataattgataatgttgcggtgcatggccagaggcagagcgacggtgctgaacacacaactggcgcaacattggtgtatatgactgccggggcactgtatccgtgttgacacggtgtgctcaccgttgctagcaaagttagggtttaatcggctattaatggtaggtgttgagttggttgagttgggatgagcctcaggatcgccgcacagggctatacgctcacacgagcaacgcgacaaatgacgtaaccttgagggttaatatgagctctgtggacgctcgttcttgttgcaaacgttctgagagaacactcacggtgtagcgatcgaagcgcgcgtgggttgttatacctgtgtccagcgctcctggcagtgcacttttgatatcagtgtgttccgtgcccccgcttcttatctgagccgcaccgcttatcccgacacaagaaaactataaagaaggctggacccccagattgctcatcatcttgccacaggaactctgagatacctgtggatatacagctttctcaggtctagactgcgcgttttctgttttattttccctttttagatcgactggattgattcctagttgatttcatttttattccgtttgtctgaacaca

SEQ ID NO: 37 - DNA, 1000 bp, Arxula adeninivorans
agctaggtcaagcgacgcctgttagcgataacgaccttgaaatatctacgcgtgggccgtgtgtcgtaactgtacagtgacgttacgaccagacaatagtggtggaggggtagccagtgggaatggagcttgagcgagagaaaaatgacatcaccgaaaaaaaggcggtgagggttttgttactggggagacgcgcgtgcgccccgtggtgtgcggcgtggggctcggcagtgccgacccatttcacccatggaatcgtctagacaggcaaaatggcgtgagcgcctgccggagatactaaagtttgcagcgaaagaaggagaacaaacgcacgaaccaaatcagagccaaattggccaggtggcaaagccaacgggcaagtccacgggcaattgcattgcccttgcccctctttggccgatactcggacatggtcgggatagaattgtgaagaacgataagctttagttaaaactgagtcattccctcatcggctaacgtgatggaggcacgtgattctccgggggtttttcgctcggtcaggctcggccgaccgtcggacggcacggcgcggtaattgtccggcccccttgtgagtgtcacctaccctgcagggcccaggcaattagtcaatcccgaggacagatggacgagaggttaggcggtattttgagaggatgttggccattgtgtagaatataaaggagactaaaaaattgcgagaatttttccgagtagaaccatgtaacttttgtctgtccaaatcggtacatttccgtgtctttgtttggaaaagctgtctctccttccctccctaagcccgaatctggggtgcagacgataaccccagaccacgaggctgcctcggccctcggatcattgacagaacaagaatgaatcacctgaaaatttggtctatataaagggccccatcccctctccatgttcgatcattaatcaaccaattggtttttaagttattgacattataaaaacaaaa

SEQ ID NO: 38 - DNA, 1000 bp, Arxula adeninivorans
gccgcgggtgtattttcaatccaataattcacagttctgagcgttgtgaatagcatctcccgataacttcaggcatcatgccacagatcagcaacccgagtacacacacgtgaccagtaggcacgtgacatccccccatttcggcatttgcgatcgttcatgtgccagcatatgaccacagagcttgtgatagtttagctccatcaggtgattttattagaattatcaacctctggagtggtcagagatggcaccaggggcacccgaagtgtagtggtgcgtgcagacatccaatgtccgaagggcttattgacccttctgccatagtgtgcaagtagagccgacgagatcggtccagcaccgctttgtcaattaattttttcccttgtaaaaaggctgcttgccattgtctcgacaaatcgactgaaaagtggcccgatttggatctcgacaatcatttgcaatcatttggagaggccacagttgtctgcggtggcattgtcatgtccccctgttgctatgtgtgccagtgactcgctccgcctgcaatttagttccccattcataccccgtaaccccggggcgtttccccagatttcctcggcaccgctcaccgaagcccttaaccccccgagtgccgaaaagtcggtattctcggaaggcatatagagaattatgaaataaaaagaggacaataaagcacgccggatacagagcgagcggtagccaaccctctaccgtcttgtcccattctctagcatcatttctccgtccgtaccttcacccaatcctacctcccggacttgtcctacgcgggtcccatcgccgagcgcagccgcacactttcacgagccgaggtccaccccccttcttcttctttgggaccacacacttcccccacattgcacatataaagctcccgaatcagccatcatacgacttcctcacaaagcctttggccggttctattttatcacaaaaccttcgataatataacaca

SEQ ID NO: 39 - DNA, 1000 bp, Arxula adeninivorans
attgggtgtggacaaagctgctagccccgagcccgaggaggatgaacaggaggattctgacaagcgtgagtatcccatgatggagaccctccctcaccctcgattcaatgctgctacatgcgtagttgatgacactctatttatctttggaggcacctatgaggatggcgagcgggagatttatctcaattccatgtatgcagttgatctaggccgtctggatggtgttagggtgttctgggaggatctacgggagctggagcaggccggctcagacgatgaagacgatgacgacgatgaagatgatgacgaagaggacgatgatggtgaagatggagaggatcacgacgaggatcaggatcaagtcgaagccgaggacgaaaaggacaatcaagaagaggaggaggaagctgaaaagagcgacatgaccattccagatcctcgaccttggctgcctcatcccaagccattcgaatcgctccgagcattctaccagcgaacgggacctcaattcctggaatgggccctgtccaaccatcgggacgctagaggaaaggacttgaagcgaattgcatttgaactgagcgaagaccgatggtgggagcgacgagaggaggttcgtatctccgaggaccagtttgaagagatgggcggagtcggtgaggtcattgaaaaggacgctcctagaaaagcacgacgataaatagactaatccatctatcggtatcaggctatgaaactatcaatctgtcaaaatctgtcaacatatcagctactaatcctacgaagcctacactaccaatcctaatcctatcaatcctatcagcctatcaagctatcaactaccaacccatcaacctaccatcctaacaaacctatcaacctatcaacctatcaacctatcaacctatcaatcctatcaacctgtcaacctaccaacccaccagcctataaaccctgtatgtgttgctccgcaatccccggtggcccgcagattaat

SEQ ID NO: 40 - DNA, 1000 bp, Arxula adeninivorans
taagtcttgtatctgttacgacgctcccagtctccgcccttgtcgatgagcagtttgaccgcctccagctcctgggccacaaacaccttgtcgtcgaaaaagaagccaatacggatgatggttagcgaaatgtcaatcttgactccggacgagcccgtgagctcaaacgcctttcgcagcgtcgaaatggcctgctcgcggtcgccaatcttagcgtagtactctccgagcttgacccagttttctacaatcgcaagctcttcctcctcctcctctgcctcggcaatcttcttctgcagctcctctacctgctgctggttctccttcttaagctcctcgtacaacgactcgtcccactccagcactcctggcagaccctcggtgtgaaggtactgatataatggggccagcttttcctttttgatttctgtcatgagcgtctttttagcctggtcatgctgcggtttgagaaacggcgtcttcagcacaaagatgcattgcgccagattataatcgggcactcggtcaattggagtcgcggctccttcgttcacacccatcctacctgtctatttactccagcagtgtgtgttagtggcaactgggaagtgtcgctggttttggtgtcgatggtgcagccgtgccgtatgagccaccactagccacaatctcccgccggtgtggcggtgctcgctctatttatacagcaaatgtgcaacacaactgtagttttgttgtaattctgccaattgcacaacaaattcacagaaaaattcacaagaatgttctactaacgtagcagtacccttggccaagtaatcgtatcgatcgatcgcaatcctgatctcaatcggtcccaattctggatcccctttaccctagtctcctcccctgctggtcccctactaccagcgtaaacaaggcggaagaccctgcgttcctctgcggtggagcaaacctctctctgtcactttcacttttttcactagcagcttgtaca

SEQ ID NO: 41 - DNA, 1000 bp, Arxula adeninivorans
gtctgagtttggtcagattttcaaaaacccatcaaaggagttcttccagaaggcagagcttcgagctgccagagcgacatggcccaagatgtcccacattcacaaccgtgtggccatcgagttggctttagtaaaggcaattcacaagcttcgtgcccgtattgtatctcagagcgtccatgagcctggcagttctctacaagtacatgctgctaatgacgaaggcaccctagcacctattcgccgtcgccattcttcgaccaagcttcaccatagacgacaacggtccgatggaatggccgtgaaatacttggtccgcagacattcgctacagtactttggcactgagggccctggtcccgctgcgctatctcgtaaaaagagttcggccgggcttacccaggctcatactcctacgccttcactgaccaacagcgttagtgtagggggcagtccaaggcaccgtcgcttcactactagctctagacagtcctcaggagaccatttggaaatgttctctcaaaatcatccgctagaacgtatctctaccggctgaccgcaacggtcttcattcatggcaattagacagctttaaattatttagaactacaaactaccaatgcatgctttacgacctttacgacctctacgaccgttaacaaccgtaacaaccttgtgtctaattatcacagtctatcacagtctattacagtccatcacagttcatgtcgtattcatctataaccttccatgacttccctcgtccctgtcgaaggccatcgaacttgcccgtagttattaatttgtccgtcatcatcaagctgcatgacccccgacgccgcacgccccggccgaccaaccatcaaaggcgataagaatgagtcaaaaaggactaaatattccggatcacgtaatcggcgcagtataaaactgagctcatccgcatatttctaggcactgaaaattccaaagactttttcaactctaatcaaaaacaaaa

SEQ ID NO: 42 - DNA, 1000 bp, Arxula adeninivorans
tcagaatgttatcgacgaggccaacaaggccacccaatcttaatgatctacgattggactttgtacgacatagggatgacgatttttagattagtaatatataaccgaagacaataaagatatttgtggattctattaacaaactcactaaaagaataggatgatacgaagcaattgaggtcccaatgcttactggagcctggggaaaaatgccagtaaggtgccagcatggcaggggtttgcggtgggtcggttaggcgcgtttggacaggggtcaggtacagcggaaagctgaccattcaacgcaaacctaataactggaatttttgtagttttattctacatgttcaattgctggttttactcaaattctgaaccatgcgagcgcttgtctacaggtcctaaagtccctacagctccgtgtatgcagcttgtcaacaggtgtgacgagcactacacgtttcagcacaattgcgttcgtaacagatttttccaaggcttactagcctgtcactattattctaccggccaaattatacactttcaagcaattacttttataattgcaactctactttgcaattgttaattgtccacgaccgtcgatgacatgggtccctaatgcgtgggggccgcgcgcacggctgggaggactcgacataataaattattgcaacaaagccaaatcaattaggtgagggctgcaacgcattggcaacgagtgaccgtatctgaccaatgtccaatctgcctactgaaagctgccattgcgtcgtatacccctgatttgtgacatatcagccattgcctccttaattgtcatgctcatatactctttctacactaaataaaccccctcacggggaacgccggcaacccgcagcataacccgagtaacgctcccaacaaatttggcacggcccggtagataccggaaaaaggctcggaaaaaaatctaaataactttgcaactgacccttcaaaggttgaacagtacatc

SEQ ID NO: 43 - DNA, 1000 bp, Arxula adeninivorans
ggagaagatgtgggatattattggtcttgtaggagcccgattagggtatgattgggaccgacaaggaaggattgtctagactagtctagcctagtccagactaggtctgtaccattacgagtcgagaactgcactctgatcttgtgctatgtacgtgtgatgtaaatgaatgacgaacaatatgacgcagacgtggatgttaatctttggatggacacatttatatgatggtggaatggtggtcgttgtgaacagtatttaacaaccagattcccacactcaacttaatacaaggactcaatggctctaaatagagctgaataagtacaaggcattgttactttataccaattgagctatccaattgagttatatcaaccgtttgacgatccataattctcagtgctgtctacctcgaataactggaactactggctccaattgaccggcccagccagtgccagacagtaccaattagtccaaccactcccatatcaccaattgaacaaatccaattcccctaccaccgttacctgtaactcaccccatttcaatttgcctgtccagcttatccagcttatccatccgggattccgtttcctttctcatcgctgttggacccccactctttccctaacacactatttactctagtacacaactaattataatactattctcacctcacctccattcctcctcactaattgccactgaacctgccacaaccaccgcaccgtaccatactaattatcctggccaatttcgccagccaattccatccacttgtctcgaatgtttacatcgctactttccctacacgcttcctcgacccgggctttgccagcgtccagcggggttcccaactagtgcacggcagacccgggtagggcccccaactaatgatacctaccgggccactctgaaaaaaagacgccgttcgagccggatttttccgttgtatttggtcagaactttttttcctactcctgtattaaca

SEQ ID NO: 44 - DNA, 1000 bp, Arxula adeninivorans
tatattgaattgatacctaatatacaatagattgtccctgggacattacacgtagacgttgaattgtcaactacagtatcgtcaacaggaagaacattctgtatgcccgaattgccattaccaatcctggtattcaattccctgtcccctgctgtctcttgctgtctctgtggtctctattcctagaatacactggccgagagttcggctcagtgcctgctcgtgatactcggtacgaagcctaaattgtccccgcatggttcgattccaactggaatcattttctggagtaaaatcctcggcccacgacaataatccgggtgacgtcatgtgaccctaggagggcaaacgccggcgtttcgcaacaaagcagccccagaaggccccgtttgaagcgccagaaccgctctccagcgagactacaacccgtactacgtatctacccgttttgtagcgatttccagcgtcaatttcatgtccttttcttcatctccagcttctccagttcactctccagcccttctgttcatctccttactccgaatcgggggatttttggcaagggttgtccgattcgtcggtcgggctgcggcttgggtgcccattaacgtgaccgaatgccgcactccccccgattgacgaaacaaaggaaagcaataactggggtaagaggagattgggtcgcaatgattgcacgagcccggacggtagcgcaattgagcaccattgtcggcggtcgactgcctgggcttctggtatgcctgcaaatgccggcagcatctccgaccaattaccgtagtgaattttgtgcagcattttttaccattaacccgatgcccgaatcggccccgacacctgcgtttgtgtaattgcgagcccatgattggttgccccggacaggcgtggctttccggccccaaagtatataacaaactgcaatcgcaaattcgcatttttttttccgtcgtctagttgcaagtccaattcgcggagatttacc

SEQ ID NO: 45 - DNA, 1000 bp, Arxula adeninivorans
tcgcaggccgctaatagaacagtgggctcatttgggcggctcaagccgcattaccactgtggcctcacggggcttacggggctcctgcggctcctgcggcgcacaaccgtgtatatatttccgctggattccacgcccacggtagtctaatccatgtaacgggttgctaaattgtctagaattgctaaacttgctaaattgctaaacttctgaacgctaaaactgctaaattgcttacttctactactgccattaacactctggctattgcttatccctatacctacctgttcttcgcttttctatagctattttcacactgcccattggtttcccattggttagaacccgagggtcccgatgccggcagccgtacaccctggcgtctttgtccaaaactgggccgtatcgcggtcagacaacaggccattctcgggtggtatgagagacggactaatgcgctagtaacatccggtctataccattgagcgcctgagtaaccacaattgcgtgactaattctgtttgcattcggttaacccctctctgctctgatactaatcgtgacggcgcggcgcaattatcgtgtttgttgggcgtcccctgtccgagatttgaaggtcccgataattatcgtcggcaaaaaccgttactataatgcatttgacggacccaaatgatgagttggcaaccgtttgcaatcacaatgaccccaaatcctgatggaaaatgccttgaaaggtacatttccacatttagtccactcccccccttggtcttgttgagcgccccactgcgtctcattcaatgctgattggttctttttgaccaaacggtggtatattatctaacgcaccatcacacaagggcgaggctagttgctacatggcatcatgctgcagatgatatatatataaagccctctcccacccgcaggaccaacaagaaaaagtttcacaccaaagtccgtgtatcttttttgtccaaaaaaaataacaaca

SEQ ID NO: 46 - DNA, 1000 bp, Arxula adeninivorans
aaaaggggagacgtcagccatcctgttagtgtcagtttgccctacatttgcgcgtccctgtgtccttttatattcctctctcctgaagccgaaaaaagtaattgcaaactaccatgcggtggggacatgatggcagataatcaccgatgatgattatcgcacaccgtgattagcggctcatgtcccatgatgtggcgctaccctgccggagcgccgaaaaacctaccgcagcagctagtttccccaggctgccacatgaaacgaggagaaatagcaatcccttggccgcgggaccagttgggggccagctgggggccattgaggtgtcattgaagtgtcattggcttgccatagaatctatccatagtagagaacgtccactttttgttcttggatatgcataagcgactccagggtgggtaaggattatccatcttctatcttggcacataggtagaagtccgcattcttgccgagtagccgacaatatatccttaagctccacaattgactttcagattagaggtttacccaagtagtaccaaggagtaccaagtagtaccaaatagtaccaactagcagttgtgaactcatataactgtttcatttggtggatggaaatcgtcaatagcggagttccatagaacggttgtataatacggaagggacacactttgttggttccattccaattgtgctagccaagcaatagtcggattgcctgcaggttaaagttagtcacgggtacagatcccgagttcagcttcgagggagtagcctcgtggcagttgtccacgagcatcaatggatcaagccacatggttttcagttctcaatttcaaagaaaccatcgcatagcatcgacttagtccaattcttgagctcttgggtgcgcatctcggtcgtggtcagggactgggaaaaaatgcgccgcatacacagcggcgtgcggccattaccttcacgcgcagagtcgcgtttgtgttgtcacgaatgacgg

SEQ ID NO: 47 - DNA, 1000 bp, Arxula adeninivorans
agcaatccaaacagtcacgtggccgttgtcaagtgaggactgcccgtgagtgcccccgccatggatgtgtcattatcacgtgactctgacaaccaagccaattgcccccgtgtctcacactcacattccagcaactgggcgccgatggagtgttacgagcggtgagtcatcagatgtgtcaactacgtacgagaacaatacacttgatcattctccgttcccctgacgtgccccttgccatggtgatagaactaaaggatggtgcggcaaacttttcctttcttctcaaaacggaaaggagtgtttcggatacgggagcgcgcgcagactccggtccggagtttgacaagactcaggggcttctgacaggccttattgtgaagaaaccagcacttttctccagtaactatcctcacaggatgccatacacgtagattagtaccaatttaccctcagtacattgctcattgagcaaactttccaattcaatctagaatgatgtccggcgattctcgccataacgggtaccggcgatctccctgcgccgcacgtgcgcctcttggacgttcggcactccgaatatccactgttttgccttgcctgtggtgcggaggatgagtaaccagtgggtacaattggctccagtttgccatcatcatgtagataagaatagaagcaaactggacagctgtagtcgccaccactagacagttgcaattgccactcacgggttctatacaccaaaccacggtctggttctgcccctttatttgaccgttgtcgttggctcttgtcctcaacaaagctcgcctacctcgcatacgaggtagcatgcgcctcacttttttaaatacgaaaaagaaattcttgggcaaatacggaaaagaaattattgggcttttcgtccccgccgatcaacgcagtgatcttgcgaagacgatatataaacagccaagagtccccgaatcataaactttttcatccgcgaattagtgctgaa

SEQ ID NO: 48 - DNA, 1000 bp, Arxula adeninivorans
ctaattcaagcccactgttgctaatctctcgacaaagcgttgagaaactgtcagaggatgctcaaacggttgtggatccatcggtattcaggggtaacattgtcatctctggtacaccagcatacaaggaagacgaatggaactatatatcaattgccggacagcggtaccggctcctgggcccttgtcgtcgttgcaacatggtatgtgtgaatggccaaggagagatcaattcggaaccctattttgcactacatcgtaccagaaagacccaaggcaaactactattcggtcagcacatgactcttgatcaacctactgattcactgaaccctgcagaagctacaattaaagtaggccaattgttcactcctatctgagacagttcacctgcagccgtgcaaactgtcaacgagggccgaatgatatggaaataatgattatgccgttatgactgtaatatgaatgaaaaaattttccttatgcattattaaagacccaaaataaacattcctgcccctgatttacaggtttatccggaaggacccggtcaaagaaaagttttccatgcgtaaaaataatattctgcgtggggggtcggctcccgactgtggccctatcaatagtgcggctgaagagcttacagaccaagctttttagctccggacaaatgaatttggtaacaagcatacaattttgttagaagtattgcgcttctttggtaattttttagtatctttagtagtctttatccaatttatgttcatttatactttgacttggccccctcgttatcttaacggtgccaggacactatcgtgcattatcggaccggatacggccgataaagcgggtcaatgtcacagttaccgattgcttacataaaagtggcgcggcgaaccgtctagaatggtggcgagtatataaggaggccatagcctagctctggacacatcacataaacaactacaaacttttacatttacacgtcgcatctacc

SEQ ID NO: 49 - DNA, 1000 bp, Arxula adeninivorans
acggcggtatccgcagctttgttgacgacaaggctctgcgatggttggcagtcaactttgcataccacgaccttctggcgtcttcggcgtgctcccgcaacactcactttccatccgcagaatacgatcacgtcatgaagcatggctacggtctggatgctctcacgggctgctgccagcctctgttcaagattctggccgagatttccgagctcgccgtcaagtggcagcgagtggacgatgcgtccttggaaaagctccgaatggtccaggtccgcgttagcgctctggagcagaagcttgaatcttgtcaccctgatcctctagacatgatctccctttctccccagcagcttgacctccaattgattctatttgacaccgtcaagattaccgccaggctccacctgcgccagtcggttctgcgtctcaatgctgcctcccttgacatgcaatgccttgtcaaacagctcaacaagaacttggagctggtgctgggtacccaggtcgaagggttggcggtgttccctctgtttgtggctggtatccattgcgtgaccacctcagacagagagctcatcaccaaacgcattgatgactactactctcgcaacctggcccgcaacatttctagagcaaaagatctcatggaggaggtgtggtctcttgatgatcacggctctcgtcacgttgattggtaccgaatcatccaggctagaggatgggatatctgttttgcctaacagctaacacgtaacgacttatgactactaactgcatatcaactatcaacaatactatccttattcaatcaactatactatctttattaagatcatctactatccttattcaaatcatctatcaactatcctcaaatttcgtctgtatgtgatccatgcacgtgacctttacccgtgaccacatcccgtgacaatacacgtgaacagttgtgccaactcagcaccaaatcccctttcgagcttaaccgacgacagca

SEQ ID NO: 50 - DNA, 1000 bp, Arxula adeninivorans
aatgattgatacaccttgttacgaccttgctgcgtggtgggcaagtaaactggaacttgatatatgcgtgccgtttatctgtcataagccaatcgtcaatcacacaatcaaatcaaaaactactgctagcatggcgaacctaaatgggcatcaatggaaattatacaaacagtacgagatgaaaacagtcagctatgtcatggtgtgatagttaccaggttcattttctgatttcctttgccagttctgtgcgcctgcctcattggatttgactctttttggcatcatgctcacctctggtgatacacgagctagactgctgaaagaagtatcagcacagccaacgaggttgcagcaaatagggcatacctgttatcgccgaccaggcattatcgaccaccagctatttgcgtctcatgcatcccatttcctgatgaagctgtgtcccgtcgattacgcctatccttctttgccaagccttaccagggacccaatcatatcgggaccctacgcaacgtgaatccggggtaggatatcgagctcccgaacgttgaaccaaattttaacggtggtgggagatcacagatcagcgacaccactataatctgcagtcgcaaccatcacagacctccgtgaagtgatatagaatcgctccagaaagactatggcaggctcgtttttcccagtgcaagagctatttcgggcgagcttctagcggctcccattgtcagaccttaattgtgctccatttaggcacgtggaggtgccaagattagtgtttgaggattctccctgttgccaagtctctaaagaagatagacagtgttaagctactgagcttggcacttgactacccaatgagaaggatgagccaacccacctgatgagtaggtatcaggtaacggttgaccatagaacgagtcaattgtggaaatataaaaagggagccaaattggattgattcaccaagaatccaataaaaaaaagaagtcactgaaaa

SEQ ID NO: 51 - DNA, 1000 bp, Arxula adeninivorans
agctcgttccaccccctttccccctgtctccaccctaaccctccggtcatactagcaccactaccgaatgagagtagcaccatgtatcataataaccgcgccagggcgacacaacattgaccgaacaatatcaatatcgaggtacaataactgcgtgtctgtgaggccagattacatgcgtctgcacgtttgtgaccgatatcaggcggcggccgataagggcaagtgaaatttcacgtggaccgtctcacgtgaacacgggatggcggcagcaatcgttggcccaccgtactggccaagcaggcccaacaataaagaaattcagtggaaaaacccagaccaggggacgcagcgcacccctgtaaccgcccggcacgcccggcgcgattgagaccaccgcagagtttttccggcacagtttttccggcctggggtgacccttgagcgcgccggaatggcccgtatcaccctactccgacagaacccggtgcggcgagctgaggcggtgggacgattgcggcggcctgcggcgcatttcgggaccgccttccttgttatgatacgattgcggcaccgtgaggcgttcctgatggttccgagattcagcgcaaccttgatgcaacaagtaatcaattcgcagccagaatggtggcaatttggtgagcaatagtaaaaaaacagtagaatataggtgtaggaaaacgtagacagtaggctttttgggtccctttagccattgtaactaaatagctggacctgcaggacaaagaccctgtacacggaacaatttaagcccttagctgtacccacaggcatcccccaccgttttaagggacgtcgcaactaacgcctaaacggaacaaggacccggaaagtcgtacgtctaatacggcaaagtgggctataaaagggggcgctactgccaacccaatgagttcatccgatcaccattgacagttgtcaattaacaatacacatccatcttgtaccctaaacaata

SEQ ID NO: 52 - DNA, 1000 bp, Arxula adeninivorans
tctaccatcaggaaactggaggggcgtcttcagtacgacaaggcggagaggtatcgtactctttggcaactgctcctaggatcgattctactcctttgtgtctatgccattactaattttttattttttatggccgaagaaccgaccctggattccagcagcacttggaagtctcgttggttcattctggaagaatttcctaatctggtttacttcgttgactttagcgttattgcctacatttggcggcccaatactaacgacgtcaggttcatgtcgtccaagattgcccaggatgagaatgaagttcaagagtttgaaattggatctctccgagagtctatggacgagtaagagatattaaggaattgaaaaagggcaagaaaagagcgatgagcgtagaaattgcgtagaaattgtagcagtatcaatacccttaccatcacctaaagcaaaccaaaagatcccgggtgaatctccgggacctgagtagatggtaatacagaatactggcagaatactgcactcagaagaactctggaagaactctggaagcagtctaacggaccccagtttggctcttgaacattcacgtgactggaaacttaacatcacgtgacctcgtccagtctggattgaaatagggctgaaataaaaaatcagtacacaatgagagtttggccgagtggtctatggcgtcagatttaggtaaaccctaaagtgaattctctgatatcttcggatgcgcgagttcgaatctcgtagctctcattatcttttttactccctttccgtttcggactaaccacggataccttttccaagcaatttgcgatccaattatttttgttcttttaattaaatttagtttcattcatctccggtcccccttgatagatgaacgtccgtatttaccgttaagccgcataaccgccaggaaagccccgatctgtcaaccttggcatctactacgtttcgtttataactctcgctcgtttta

SEQ ID NO: 53 - DNA, 1000 bp, Arxula adeninivorans
gcccagtgcattgtccttgtcattctaggagtggcctttttcattgcatttgtacttgtagaacgagctgtggacacccctctagtaccggttcgcaagtttaacactaatatggccagagtgctcgcttgtgtggcctttggatggggcacttttggtatctggatttactacctttggcagattatggaatacctgcgacacaactccccattgttggcttcagctcagttctctccagctgccgcgatgggtgccattgctgcaattgctactggatacctcatgtcaaagctacatcctttccgagtgctggcaatttccctgttggcgttcctggtcgcttcaattatcaccgccacggcgcctgtaaaccaaacgttctgggctcagacgtttgtatcaatcttagtagcttcttggggtatggacatgaacttccctgctgcgacccttatcttatcagagaccgtgcccagggaacagcagggaattgccgcctctttagtggccactgtggtcaattattcaatctccctaagcctgggagttgcaggtactatcattgagcaggtatctccaggtttggaccctaattcatatttaaagggcgtccgaagcgccctatatttctgcattggcctctctgccgccggccttcttgtcgctctctatggtgtcatcagagacgacattcttgctaaccatgggaaatcctctaacgacgaagaaaagaatactgcttgaaatgcttttttaatagaattttgctcttatttgtcctatttaatctatatttcatgtacgaatcgatttctaatcttaacaccgcggagattcttttgttattactaaatcaggaaaagatgcacggagaactcggcccgagttggatttgatggcatctcggtccgagttaaacgtggggtaatcttttagcggggaaagttataaaacccctacaaagcccaggatttgtgaattcacatttgacaacaca

(19) United States
(12) Patent Application Publication
(10) Pub. No.: US 2017/0211078 A1
(43) Pub. Date: Jul. 27, 2017

(54) Promoters derived from Yarrowia lipolytica and Arxula adeninivorans, and methods of use thereof

(71) Applicant: Novogy, Inc., Cambridge, MA (US)

(72) Inventors: Annapurna KAMINENI, Arlington, MA (US); Elena E BREVNOVA, Belmont, MA (US)

(21) Appl. No.: 15/328,835

(22) PCT Filed: Jul. 24, 2015
(86) PCT No.: PCT/US2015/041910

§ 371 Date: Jan. 24, 2017

Related U.S. Application Data
(60) Provisional application No. 62/028,946, filed on Jul. 25, 2014.

(51) Int. Cl. C12N 15/81; C12N 9/26; C12N 9/10

(52) U.S. Cl. CPC C12N 15/815; C12N 9/1029; C12N 9/2431; C12Y 203/0102; C12Y 302/01026

(57) ABSTRACT

Disclosed are the nucleotide sequences of promoters from Arxula adeninivorans and Yarrowia lipolytica which may be used to drive gene expression in a cell. The promoters were validated, and selected promoters were screened to determine which promoters may be useful for increasing the lipid production efficiency of oleaginous yeasts.

### RELATED APPLICATIONS

This application claims the benefit of priority to U.S. Provisional Patent Application No. 62/028,946, filed Jul. 25, 2014, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jul. 16, 2015, is named NGX_03425_SL.txt and is 71,975 bytes in size.

### BACKGROUND

Oleaginous yeasts, such as Yarrowia lipolytica and Arxula adeninivorans, may be engineered for the industrial production of lipids, which are indispensable ingredients in the food and cosmetics industries, and important precursors in the biodiesel and biochemical industries. The lipid yield of an oleaginous organism can be increased by up-regulating or down-regulating the genes that regulate cellular metabolism and lipid pathways.

One approach to up-regulating a gene is to control its expression using a strong constitutive promoter. For example, the Y. lipolytica diacylglycerol acyltransferase DGA1 may be up-regulated using a strong constitutive promoter, and such genetic engineering significantly increases the organism's lipid yield and productivity (See, e.g., Tai & Stephanopoulos, METABOLIC ENGINEERING 12:1-9 (2013)).

Choosing optimal promoters for controlling gene expression is a critical part of genetic engineering, but different promoters may be optimal for different applications. For example, the optimal promoters for an industrial strain of yeast may not be the same as promoters that are optimal in laboratory strains.

Some Y. lipolytica and A. adeninivorans promoters have been identified and validated (See, e.g., U.S. Pat. No. 7,259,255 (incorporated by reference) and U.S. Pat. No. 7,264,949 (incorporated by reference); U.S. Patent Application Nos. 2012/0289600 (incorporated by reference), 2006/0094102 (incorporated by reference), and 2003/0186376 (incorporated by reference); Wartmann et al., FEMS YEAST RESEARCH 2:363-69 (2002)). Both organisms, however, contain hundreds of promoters that have yet to be identified, and many of these promoters could be useful for engineering yeast and other organisms. Further, a promoter may vary considerably between different strains of the same species, and the identification and screening of such genetic polymorphisms provides a richer toolbox for genetic engineering.

### SUMMARY

Disclosed are the nucleotide sequences of Arxula adeninivorans and Yarrowia lipolytica promoters that may be utilized to drive gene expression in a cell. These promoters were validated, and selected promoters were screened to determine which may be useful for increasing the lipid production efficiency of oleaginous yeasts.

### BRIEF DESCRIPTION OF THE FIGURES

FIG. 1 depicts a map of the pNC303 construct, which was used as a template to amplify a DNA fragment comprising the Saccharomyces cerevisiae invertase gene SUC2 and the TER1 terminator. “Sc URA3” denotes the S. cerevisiae URA3 auxotrophic marker for selection in yeast; “2u ori” denotes the S. cerevisiae origin of replication from the 2 μm circle plasmid; “pMB1 ori” denotes the E. coli pMB1 origin of replication from the pBR322 plasmid; “AmpR” denotes the bla gene used as a marker for selection with ampicillin; “ScFBA1p” denotes the S. cerevisiae FBA1 promoter −822 to −1; “hygR(NG4)” denotes the Escherichia coli hygR gene cDNA synthesized by GenScript (SEQ ID NO:2); “ScFBA1t” denotes the S. cerevisiae FBA1 terminator 205 bp after stop; “Y1TEF1p(PR3)” denotes the Y. lipolytica TEF1 promoter −406 to +125; “NG102” denotes the S. cerevisiae SUC2 gene (SEQ ID NO:1); “Y1CYC1t(TER1)” denotes the Y. lipolytica CYC1 terminator 300 bp after the stop codon.

FIG. 2 depicts the invertase activity of Y. lipolytica strain NS18 transformants expressing the Saccharomyces cerevisiae invertase gene SUC2 under the control of 14 different promoters and the same TER1 terminator (Y. lipolytica CYC1 terminator 300 bp after the stop codon). The x-axis labels correspond to Promoter IDs in Table II. Activity was measured by a dinitrosalicylic acid (DNS) assay. Samples were analyzed after 48 hours of cell growth in YPD media in 96-well plates at 30′C. The samples in 2A and 2B were analyzed in different 96-well plates. The parent Y. lipolytica strain NS18 (“C”) was used as negative control on each plate.

FIG. 3 depicts a map of the pNC161 construct used to express the hygromycin resistance gene (hygR, SEQ ID NO:2) in Y. lipolytica strain NS18 and A. adeninivorans strain NS252. Vector pNC161 was linearized by a PacI/PmeI restriction digest before transformation. “pMB1 ori” denotes the E. coli pMB1 origin of replication from the pBR322 plasmid; “AmpR” denotes the bla gene used as a marker for selection with ampicillin; “Sc URA3” denotes the S. cerevisiae URA3 auxotrophic marker for selection in yeast; “2u ori” denotes the S. cerevisiae origin of replication from the 2 μm circle plasmid; “ScFBA1p” denotes the S. cerevisiae FBA1 promoter −822 to −1; “hygR(NG4)” denotes the Escherichia coli hygR gene cDNA synthesized by GenScript (SEQ ID NO:2); “ScFBA1t” denotes the S. cerevisiae FBA1 terminator 205 bp after the stop codon.

FIG. 4 depicts agar plates with A. adeninivorans strain NS252 transformants expressing the Escherichia coli hygromycin resistance gene (SEQ ID NO:2) under the control of different A. adeninivorans promoters. The labels correspond to Promoter IDs in Table I. The transformants were grown for 2 days at 37° C. on plates containing YPD and 300 μg/μL hygromycin B. The negative control consists of the parent A. adeninivorans strain NS252 transformed with water instead of DNA.

FIG. 5 depicts agar plates with Y. lipolytica strain NS18 transformants expressing the Escherichia coli hygromycin resistance gene (SEQ ID NO:2) under the control of different A. adeninivorans promoters. The labels correspond to Promoter IDs in Table I. The transformants were grown for 2 days at 37° C. on plates containing YPD and 300 μg/μL hygromycin B. The negative control consists of the parent Y. lipolytica strain NS18 transformed with water instead of DNA.

FIG. 6 depicts a map of the pNC336 construct used to overexpress the gene encoding diacylglycerol acyltransferase DGA1 (SEQ ID NO:3) in Y. lipolytica strain NS18. Vector pNC336 was linearized by a PacI/NotI restriction digest before transformation. “Sc URA3” denotes the S. cerevisiae URA3 auxotrophic marker for selection in yeast; “2u ori” denotes the S. cerevisiae origin of replication from the 2 μm circle plasmid; “pMB1 ori” denotes the E. coli pMB1 origin of replication from the pBR322 plasmid; “AmpR” denotes the bla gene used as a marker for selection with ampicillin; “PR14 AaTEF1p” denotes the A. adeninivorans TEF1 promoter −427 to −1 (SEQ ID NO:5); NG66 (Rt DGA1) denotes the Rhodosporidium toruloides DGA1 cDNA synthesized by GenScript (SEQ ID NO:3); “Y1CYC1t(TER1)” denotes the Y. lipolytica CYC1 terminator 300 bp after the stop codon; “ScTEF1p” denotes the S. cerevisiae TEF1 promoter −412 to −1; “NAT” denotes the Streptomyces noursei Nat1 gene used as marker for selection with nourseothricin; “ScCYC1t” denotes the S. cerevisiae CYC1 terminator 275 bp after the stop codon.

FIG. 7 depicts lipid assay results for Y. lipolytica strain NS18 transformants expressing the Rhodosporidium toruloides DGA1 protein under the control of different A. adeninivorans promoters and the same TER1 terminator (Y. lipolytica CYC1 terminator 300 bp after the stop codon). The x-axis labels correspond to Promoter IDs in Table I. For each construct, 12 transformants were analyzed by the lipid assay described in Example 7. The samples were analyzed after 72 hours of cell growth in a 96-well plate containing lipid-production-inducing media. Sample “C” depicts the parent strain NS18 as a control, and the error bars depict one standard deviation obtained from three different assays.

FIG. 8 depicts lipid assay results for Y. lipolytica strain NS18 transformants expressing Rhodosporidium toruloides DGA1 under the control of different Y. lipolytica promoters and the same TER1 terminator (Y. lipolytica CYC1 terminator 300 bp after the stop codon). The x-axis labels correspond to Promoter IDs in Table II. For each construct, 12 transformants were analyzed by the lipid assay described in Example 7. The samples were analyzed after 72 hours of cell growth in a 96-well plate containing lipid-production-inducing media. Sample “C” depicts the parent strain NS18 as a control, and the error bars depict one standard deviation obtained from three different assays.

FIG. 9 depicts a map of the pNC378 construct used to overexpress the gene encoding diacylglycerol acyltransferase DGA1 from Rhodosporidium toruloides in A. adeninivorans strain NS252. Vector pNC378 was linearized by a PmeI/AscI restriction digest before transformation. “Sc URA3” denotes the S. cerevisiae URA3 auxotrophic marker for selection in yeast; “2u ori” denotes the S. cerevisiae origin of replication from the 2 μm circle plasmid; “pMB1 ori” denotes the E. coli pMB1 origin of replication from the pBR322 plasmid; “AmpR” denotes the bla gene used as a marker for selection with ampicillin; “PR26 AaPGK1p” denotes the A. adeninivorans PGK1 promoter −524 to −1 (SEQ ID NO:14); “PR25 AaADH1p” denotes the A. adeninivorans ADH1 promoter −877 to −1 (SEQ ID NO:13); “NG66 (Rt DGA1)” denotes the Rhodosporidium toruloides DGA1 cDNA; “ScFBA1t(TER6)” denotes the Saccharomyces cerevisiae terminator 205 bp after the stop codon; “NAT” denotes the Streptomyces noursei Nat1 gene used as marker for selection with nourseothricin; “AaCYC1t” denotes the A. adeninivorans CYC1 terminator 301 bp after the stop codon.

FIG. 10 depicts lipid assay results for A. adeninivorans strain NS252 transformants expressing different DGA proteins from various host organisms under the control of the A. adeninivorans promoter ADH1 and the TER16 terminator (A. adeninivorans CYC1 terminator 301 bp after the stop codon). The x-axis labels correspond to DGA genes in Table III. For each construct, 8 transformants were analyzed by the lipid assay described in Examples 7 and 8. The samples were analyzed after 72 hours of cell growth in a 96-well plate containing lipid-production-inducing media. Sample “C” depicts the parent strain NS252 as a control, and the error bars depict one standard deviation obtained from eight different assays.

FIG. 11 depicts lipid assay results for A. adeninivorans strain NS252 transformants expressing different DGA proteins from various host organisms under the control of the A. adeninivorans promoter ADH1 and the TER16 terminator (A. adeninivorans CYC1 terminator 301 bp after the stop codon). The x-axis labels correspond to DGA genes in Table III. For each construct, 8 transformants were analyzed by the lipid assay described in Examples 7 and 8. The samples were analyzed after 72 hours of cell growth in a 96-well plate containing lipid-production-inducing media. Sample “C” depicts the parent strain NS252 as a control, and the error bars depict one standard deviation obtained from eight different assays.

FIG. 12 depicts lipid assay results for A. adeninivorans strain NS252 transformants expressing different DGA proteins from various host organisms under the control of the A. adeninivorans promoter ADH1 and the TER16 terminator (A. adeninivorans CYC1 terminator 301 bp after the stop codon). The x-axis labels correspond to DGA genes in Table III. For each construct, 8 transformants were analyzed by the lipid assay described in Examples 7 and 8. The samples were analyzed after 72 hours of cell growth in a 96-well plate containing lipid-production-inducing media. Sample “C” depicts the parent strain NS252 as a control, and the error bars depict one standard deviation obtained from eight different assays.

### DETAILED DESCRIPTION

### Overview

In some aspects, the invention relates to vectors, comprising a nucleotide sequence encoding a promoter derived from Arxula adeninivorans or Yarrowia lipolytica, wherein the vector is a plasmid. In some aspects, the invention relates to vectors, comprising a nucleotide sequence encoding a promoter derived from Arxula adeninivorans or Yarrowia lipolytica, wherein the vector is a linear DNA fragment.

In certain aspects, the invention relates to a transformed cell, comprising a genetic modification, wherein the genetic modification is transformation with a nucleic acid encoding a promoter derived from Arxula adeninivorans or Yarrowia lipolytica.

In other aspects, the invention relates to methods of expressing a gene in a cell, comprising transforming a parent cell with a nucleic acid encoding a promoter derived from Arxula adeninivorans or Yarrowia lipolytica. In some embodiments, the nucleic acid comprises the gene, and the gene and the promoter are operably linked. In other embodiments, the nucleic acid is designed so that the promoter becomes operably linked to the gene after transformation of the parent cell.

### Definitions

The articles “a” and “an” are used herein to refer to one or to more than one (i.e., to at least one) of the grammatical object of the article. By way of example, “an element” means one element or more than one element.

The term “DGAT2” refers to a gene that encodes a type 2 diacylglycerol acyltransferase protein, such as a gene that encodes a DGA1 protein.

“Diacylglyceride,” “diacylglycerol,” and “diglyceride,” are esters comprised of glycerol and two fatty acids.

The terms “diacylglycerol acyltransferase” and “DGA” refer to any protein that catalyzes the formation of triacylglycerides from diacylglycerol. Diacylglycerol acyltransferases include type 1 diacylglycerol acyltransferases (DGA2), type 2 diacylglycerol acyltransferases (DGA1), and all homologs that catalyze the above-mentioned reaction.

The terms “diacylglycerol acyltransferase, type 2” and “type 2 diacylglycerol acyltransferases” refer to DGA1 and DGA1 orthologs.

The term “domain” refers to a part of the amino acid sequence of a protein that is able to fold into a stable three-dimensional structure independent of the rest of the protein.

“Dry weight” and “dry cell weight” mean weight determined in the relative absence of water. For example, reference to oleaginous cells as comprising a specified percentage of a particular component by dry weight means that the percentage is calculated based on the weight of the cell after substantially all water has been removed.

The term “encode” refers to nucleotide sequences (a) that code for an amino acid sequence, (b) that can bind a protein, such as a polymerase or transcription factor, (c) that regulate proteins that bind to nucleic acids, such as a transcription start site, and (d) complements of the nucleotide sequences described in (a), (b), and (c). For example, a nucleotide sequence may encode a gene, which codes for an amino acid sequence, and/or a promoter, which binds a polymerase. Both DNA and RNA may encode a gene. Both DNA and RNA may encode a protein.

The term “endogenous” refers to anything that exists in a natural, untransformed cell i.e., everything that has not been introduced into the cell. An “endogenous nucleic acid” is a nucleic acid that exists in a natural, untransformed cell, such as a chromosome or mRNA that is transcribed from naturally-occurring genes in the chromosome. Endogenous nucleic acids include endogenous genes and endogenous promoters. The terms “endogenous gene” and “endogenous promoter” refer to nucleotide sequence that naturally occur in a cell's genome, which have not been introduced by transformation or transfection.

The term “exogenous” refers to anything that is introduced into a cell. An “exogenous nucleic acid” is a nucleic acid that entered a cell through the cell membrane. An exogenous nucleic acid may contain a nucleotide sequence that did not previously exist in the native genome of a cell and/or a nucleotide sequence that already existed in the genome but was reintroduced into the genome, for example, by transformation with an additional copy of the nucleotide sequence. Exogenous nucleic acids include exogenous genes and exogenous promoters. An “exogenous gene” is a nucleotide sequence that has been introduced into a cell (e.g., by transformation/transfection) and encodes an RNA and/or protein, and an exogenous gene is also referred to as a “transgene.” Similarly, an “exogenous promoter” is a nucleotide sequence that has been introduced into a cell (e.g., by transformation/transfection) and that encodes a promoter. A cell comprising an exogenous gene or an exogenous promoter may be referred to as a recombinant cell, into which additional exogenous gene(s) or promoter(s) may be introduced. The exogenous gene or exogenous promoter may be from the same species or different species relative to the cell being transformed. Thus, an exogenous gene can include a gene that occupies a different location in the genome of the cell than an endogenous gene or is under different operable linkage, relative to the endogenous copy of the gene. Similarly, an exogenous promoter can include a promoter that occupies a different location in the genome of the cell than the endogenous promoter or a promoter that is operably linked to a different gene than the endogenous promoter. An exogenous gene or an exogenous promoter may be present in more than one copy in the cell. An exogenous gene or an exogenous promoter may be maintained in a cell as an insertion into the genome (nuclear or plastid) or as an episomal molecule.

The term “expression” refers to the amount of a nucleic acid or amino acid sequence (e.g., peptide, polypeptide, or protein) in a cell. The increased expression of a gene refers to the increased transcription of that gene. The increased expression of an amino acid sequence, peptide, polypeptide, or protein refers to the increased translation of a nucleic acid encoding the amino acid sequence, peptide, polypeptide, or protein.

The term “gene,” as used herein, may encompass genomic sequences that contain introns, particularly polynucleotide sequences encoding polypeptide sequences involved in a specific activity. The term further encompasses synthetic nucleic acids that did not derive from genomic sequence. In certain embodiments, the genes lack introns, as they are synthesized based on the known DNA sequence of cDNA and protein sequence. In other embodiments, the genes are synthesized, non-native cDNA wherein the codons have been optimized for expression in Y. lipolytica or A. adeninivorans based on codon usage. The term can further include nucleic acid molecules comprising upstream, downstream, and/or intron nucleotide sequences, including promoters.

The term “genetic modification” refers to the result of a transformation. Every transformation causes a genetic modification by definition.

The term “homolog”, as used herein, refers to (a) peptides, oligopeptides, polypeptides, proteins, and enzymes having amino acid substitutions, deletions and/or insertions relative to the unmodified protein in question and having similar biological and functional activity as the unmodified protein from which they are derived, and (b) nucleic acids having nucleotide substitutions, deletions and/or insertions relative to the unmodified nucleic acid in question and having similar biological and functional activity as the unmodified nucleic acid from which they are derived. For example, a Y. lipolytica may be homologous to an A. adeninivorans promoter that is regulated by the same transcription regulators.

The term “integrated” refers to a nucleic acid that is maintained in a cell as an insertion into the genome of the cell, such as insertion into a chromosome, including insertions into a plastid genome.

“In operable linkage” is a functional linkage between two nucleic acid sequences, such a control sequence (typically a promoter) and the linked sequence (typically a sequence that encodes a protein, also called a coding sequence). A promoter is in operable linkage (or “operably linked”) with a gene if it can mediate transcription of the gene.

The term “native” refers to the composition of a cell or parent cell prior to a transformation event.

The terms “nucleic acid” refers to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “parent cell” refers to every cell from which a cell descended. The genome of a cell is comprised of the parent cell's genome and any subsequent genetic modifications to its genome.

As used herein, the term “plasmid” refers to a circular DNA molecule that is physically separate from an organism's genomic DNA. Plasmids may be linearized before being introduced into a host cell (referred to herein as a linearized plasmid). Linearized plasmids may not be self-replicating, but may integrate into and be replicated with the genomic DNA of an organism.

A “promoter” is a nucleic acid control sequence that directs transcription of a nucleic acid. As used herein, a promoter includes necessary nucleic acid sequences near the start site of transcription. A promoter also optionally includes distal enhancer or repressor elements, which can be located as much as several thousand base pairs from the start site of transcription.

“Recombinant” refers to a cell, nucleic acid, protein, or vector, which has been modified due to the introduction of an exogenous nucleic acid or the alteration of a native nucleic acid. Thus, e.g., recombinant cells can express genes that are not found within the native (non-recombinant) form of the cell or express native genes differently than those genes are expressed by a non-recombinant cell. Recombinant cells can, without limitation, include recombinant nucleic acids that encode for a gene product or for suppression elements such as mutations, knockouts, antisense, interfering RNA (RNAi), or dsRNA that reduce the levels of active gene product in a cell. A “recombinant nucleic acid” is derived from nucleic acid originally formed in vitro, in general, by the manipulation of nucleic acid, e.g., using polymerases, ligases, exonucleases, and endonucleases, or otherwise is in a form not normally found in nature. Recombinant nucleic acids may be produced, for example, to place two or more nucleic acids in operable linkage Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Additionally, a recombinant nucleic acid refers to nucleotide sequences that comprise an endogenous nucleotide sequence and an exogenous nucleotide sequence; thus, an endogenous gene that has undergone recombination with an exogenous promoter is a recombinant nucleic acid. A “recombinant protein” is a protein made using recombinant techniques, i.e., through the expression of a recombinant nucleic acid.

The term “regulatory region” refers to nucleotide sequences that affect the transcription or translation of a gene but do not encode an amino acid sequence. Regulatory regions include promoters, operators, enhancers, and silencers.

The term “subsequence” refers to a consecutive nucleotide sequence found within a nucleotide sequence that is less than the full-length nucleotide sequence. For example, a subsequence may consist of 100 consecutive nucleotides selected from the nucleotide sequence set forth in SEQ ID NO:5, which is 427 nucleotides long; 328 subsequences of 100 consecutive nucleotides may be found in a sequence that is 427 nucleotides long. A subsequence that consists of 100 consecutive nucleotides at the 3′-terminus of a full-length nucleotide sequence refers to the final 100 nucleotides found in that sequence. For example, a subsequence may consist of 100 consecutive nucleotides at the 3′-terminus of SEQ ID NO:5, and this subsequence is the final 100 nucleotides of SEQ ID NO:5. In other words, 100 consecutive nucleotides at the 3′-terminus of SEQ ID NO:5 is the nucleotide sequence of SEQ ID NO:5 with the first 327 nucleotides deleted, which is a single subsequence. As used herein, a subsequence consists of at least fifty nucleotides.

“Transformation” refers to the transfer of a nucleic acid into a host organism or the genome of a host organism, resulting in genetically stable inheritance. Host organisms containing the transformed nucleic acid fragments are referred to as “recombinant”, “transgenic” or “transformed” organisms. Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. Typically, expression vectors include, for example, one or more cloned genes under the transcriptional control of 5′ and 3′ regulatory sequences and a selectable marker. Such vectors also can contain a promoter regulatory region (e.g., a regulatory region controlling inducible or constitutive, environmentally- or developmentally-regulated, or location-specific expression), a transcription initiation start site, a ribosome binding site, a transcription termination site, and/or a polyadenylation signal. Alternatively, a cell may be transformed with a single genetic element, such as a promoter, which may result in genetically stable inheritance upon integrating into the host organism's genome, such as by homologous recombination.

The term “transformed cell” refers to a cell that has undergone a transformation. Thus, a transformed cell comprises the parent's genome and an inheritable genetic modification.

The terms “triacylglyceride,” “triacylglycerol,” “triglyceride,” and “TAG” are esters comprised of glycerol and three fatty acids.

The term “vector” refers to the means by which a nucleic acid can be propagated and/or transferred between organisms, cells, or cellular components. Vectors include plasmids, linear DNA fragments, viruses, bacteriophage, pro-viruses, phagemids, transposons, and artificial chromosomes, and the like, that may or may not be able to replicate autonomously or integrate into a chromosome of a host cell.

### Microbe Engineering

### A. Overview

Exogenous promoters and genes may be introduced into many different host cells. Suitable host cells are microbial hosts that can be found broadly within the fungal families. Examples of suitable host strains include but are not limited to fungal or yeast species, such as Arxula, Aspegillus, Aurantiochytrium, Candida, Claviceps, Cryptococcus, Cunninghamella, Hansenula, Kluyveromyces, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, and Yarrowia. Yarrowia lipolytica and Arxula adeninivorans are well-suited for use as the host microorganism because they can accumulate a large percentage of their weight as triacylglycerols.

The microbes of the present invention are genetically engineered to contain exogenous promoters, which may be strong or weak promoters. Strong promoters drive considerable transcription of an operably-linked gene. Weak promoters may nevertheless be valuable for many applications. For example, a weak promoter may be preferable to drive the transcription of either a gene that encodes a protein that displays toxicity at high concentrations or a nucleotide sequence encoding an interfering RNA directed against an essential protein. Thus, a weak promoter is preferable for expressing proteins when a strong promoter would produce a lethal amount of a protein product. Similarly, a weak promoter is preferable for expressing an interfering RNA when basal levels of the target are necessary for cell survival.

Microbial expression systems and expression vectors are well known to those skilled in the art. Any such expression vector could be used to introduce the instant promoters into an organism. The promoters may be introduced into appropriate microorganisms via transformation techniques to direct the expression of an operably-linked gene. For example, a promoter can be cloned in a suitable plasmid, and a parent cell can be transformed with the resulting plasmid. This approach can be used to drive the expression of a gene that is either operably linked to the promoter or that becomes operably linked to the promoter following the transformation event. The plasmid is not particularly limited so long as it renders a desired promoter inheritable to the microorganism's progeny.

Vectors or cassettes useful for the transformation of suitable host cells are well known in the art. Typically the vector or cassette contains a gene, sequences directing transcription and translation of a relevant gene including the promoter, a selectable marker, and sequences allowing autonomous replication or chromosomal integration. Suitable vectors comprise a region 5′ of the gene harboring the promoter and other transcriptional initiation controls and a region 3′ of the DNA fragment which controls transcriptional termination. It is preferred when both control regions are derived from genes homologous to the transformed host cell or from closely related species, although it is to be understood that such control regions need not be derived from the genes native to the specific species chosen as a production host. For example, an Arxula adeninivorans promoter may be used to drive expression in other species of yeast.

Promoters, cDNAs, and 3′UTRs, as well as other elements of the vectors, can be generated through cloning techniques using fragments isolated from native sources (Green & Sambrook, Molecular Cloning: A Laboratory Manual, (4th ed., 2012); U.S. Pat. No. 4,683,202; incorporated by reference). Alternatively, elements can be generated synthetically using known methods (Gene 164:49-53 (1995)).

### B. Promoter Sequences

In some embodiments, the invention relates to a promoter. In some embodiments, the promoter comprises a nucleotide sequence set forth in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. Promoters may comprise conservative substitutions, deletions, and/or insertions while still functioning to drive transcription. Thus, a promoter sequence may comprise a nucleotide sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53.

To determine the percent identity of two nucleotide sequences, the sequences can be aligned for optimal comparison purposes (e.g., gaps can be introduced in one or both of a first and a second nucleotide sequence for optimal alignment and non-identical sequences can be disregarded for comparison purposes). The nucleotides at corresponding nucleotide positions can then be compared. When a position in the first sequence is occupied by the same nucleotide as the corresponding position in the second sequence, then the molecules are identical at that position (as used herein nucleotide “identity” is equivalent to nucleotide “homology”). The percent identity between the two sequences is a function of the number of identical positions shared by the sequences, taking into account the number of gaps, and the length of each gap, which need to be introduced for the optimal alignment of the two sequences.

The comparison of sequences and determination of percent identity between two sequences can be accomplished using a mathematical algorithm. Exemplary computer programs which can be used to determine identity between two nucleotide sequences include, but are not limited to, the suite of BLAST programs, e.g., BLASTN, MEGABLAST, and Clustal programs, e.g., ClustalW, ClustalX, and Clustal Omega.

Sequence searches are typically carried out using the BLASTN program, when evaluating a given nucleotide sequence relative to nucleotide sequences in the GenBank DNA Sequences and other public databases. An alignment of selected sequences in order to determine “% identity” between two or more sequences is performed using for example, the CLUSTAL-W program.

The abbreviation used throughout the specification to refer to nucleic acids comprising and/or consisting of nucleotide sequences are the conventional one-letter abbreviations. Thus when included in a nucleic acid, the naturally occurring encoding nucleotides are abbreviated as follows: adenine (A), guanine (G), cytosine (C), thymine (T) and uracil (U). Also, the nucleotide sequences presented herein is the 5′→3′ direction.

As used herein, the term “complementary” and derivatives thereof are used in reference to pairing of nucleic acids by the well-known rules that A pairs with T or U and C pairs with G. Complement can be “partial” or “complete”. In partial complement, only some of the nucleotides are matched according to the base pairing rules; while in complete or total complement, all the bases are matched according to the pairing rule. The degree of complementarity between the nucleic acid strands may have significant an effect on the efficiency and strength of hybridization between two nucleic acid strands as is well known in the art. The efficiency and strength of hybridization depends upon the detection method.

The full nucleotide sequence of a promoter is not necessary to drive transcription, and sequences shorter than the promoter's full nucleotide sequence can drive transcription of an operably-linked gene. The minimal portion of a promoter, termed the core promoter, includes a transcription start site, a binding site for a RNA polymerase, and a binding site for a transcription factor. The RNA polymerase binds to the 3′-terminus of a promoter. Thus, a promoter may comprise a nucleotide sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53.

Additionally, two promoters may be combined. For example, the region of a first promoter that binds an RNA polymerase may be combined with a region of a second promoter that binds one or more transcription factors to create a hybrid promoter. Thus, a subsequence of a promoter may be combined with another promoter to change the transcription factors that regulate the transcription of an operably-linked gene. Thus, a promoter may comprise a nucleotide sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53.

### C. Vectors and Vector Components

Vectors for the transformation of microorganisms in accordance with the present invention can be prepared by known techniques familiar to those skilled in the art in view of the disclosure herein. A vector typically contains one or more genes, in which each gene codes for the expression of a desired product (the gene product) and is operably linked to one or more control sequences that regulate gene expression (i.e., a promoter), or the vector targets a gene, control sequence, or other nucleotide sequence to a particular location in the recombinant cell.

Any nucleic acid vector may encode a promoter. A plasmid may be a convenient vector because plasmids may be manipulated and replicated in bacterial hosts. In some embodiments, a linear DNA molecule may be a preferable vector, for example, to eliminate plasmid nucleotide sequences prior to transformation. Linear DNA may be obtained from the restriction digest of a plasmid or by PCR amplification. PCR may be used to generate a linear DNA vector by amplifying plasmid DNA, genomic DNA, synthetic DNA, or any other template. For example, PCR may be used to generate a linear DNA vector from overlapping oligonucleotide fragments. Suitable vectors are not limited to DNA; for example, the RNA of a retroviral vector may be utilized to transform a cell with a desired promoter.

The vector may comprise both the promoter and a gene such that the promoter and gene are operably linked. Alternatively, the vector may be designed so that the promoter becomes operably linked to a gene after transformation of the parent cell. For example, a first vector containing the promoter may be designed to recombine with a second vector containing a gene such that successful transformation and recombination events cause the promoter and gene to become operably linked in a host cell. Alternatively, a vector containing the promoter may be designed to recombine with a gene in the genome of the host cell. In this embodiment, the exogenous promoter replaces an endogenous promoter.

### 1. Control Sequences

Control sequences are nucleic acids that regulate the expression of a coding sequence or direct a gene product to a particular location in or outside a cell. Control sequences that regulate expression include, for example, promoters that regulate the transcription of a coding sequence and terminators that terminate the transcription of a coding sequence. Another control sequence is a 3′ untranslated sequence located at the end of a coding sequence that encodes a polyadenylation signal. Control sequences that direct gene products to particular locations include those that encode signal peptides, which direct the protein to which they are attached to a particular location in or outside the cell.

Thus, an exemplary vector design for the expression of a promoter in a microbe contains a coding sequence for a desired gene product (for example, a selectable marker, or an enzyme) in operable linkage with a promoter active in yeast. Alternatively, if the vector does not contain a gene in operable linkage with a promoter, the promoter can be transformed into the cells such that it becomes operably linked to an endogenous gene at the point of vector integration.

The promoter used to express a gene can be the promoter naturally linked to that gene or a different promoter.

The inclusion of a termination region control sequence is optional, and if employed, the choice is primarily one of convenience, as termination regions are relatively interchangeable. The termination region may be native to the transcriptional initiation region (the promoter), may be native to the DNA sequence of interest, or may be obtainable from another source (See, e.g., Chen & Orozco, Nucleic Acids Research 16:8411 (1988)).

### 2. Genes

Typically, a gene includes a promoter, coding sequence, and termination control sequences. When assembled by recombinant DNA technology, a gene may be termed an expression cassette and may be flanked by restriction sites for convenient insertion into a vector that is used to introduce the recombinant gene into a host cell. The expression cassette can be flanked by DNA sequences from the genome or other nucleic acid target to facilitate stable integration of the expression cassette into the genome by homologous recombination. Alternatively, the vector and its expression cassette may remain unintegrated (e.g., an episome), in which case, the vector typically includes an origin of replication, which is capable of providing for replication of the vector DNA.

A common gene present on a vector is a gene that codes for a protein, the expression of which allows the recombinant cell containing the protein to be differentiated from cells that do not express the protein. Such a gene, and its corresponding gene product, is called a selectable marker or selection marker. Any of a wide variety of selectable markers can be employed in a transgene construct useful for transforming the organisms of the invention.

For optimal expression of a recombinant protein, it is beneficial to employ coding sequences that produce mRNA with codons optimally used by the host cell to be transformed. Thus, proper expression of transgenes can require that the codon usage of the transgene matches the specific codon bias of the organism in which the transgene is being expressed. The precise mechanisms underlying this effect are many, but include the proper balancing of available aminoacylated tRNA pools with proteins being synthesized in the cell, coupled with more efficient translation of the transgenic messenger RNA (mRNA) when this need is met. When codon usage in the transgene is not optimized, available tRNA pools are not sufficient to allow for efficient translation of the transgenic mRNA resulting in ribosomal stalling and termination and possible instability of the transgenic mRNA.

### D. Homologous Recombination

Homologous recombination may be used to substitute one nucleotide sequence with a different nucleotide sequence. Thus, homologous recombination may be used to substitute all or part of an endogenous promoter that drives the expression of a gene in an organism with all or part of an exogenous promoter. Additionally, homologous recombination may be used to combine two nucleic acids that contain a homologous nucleotide sequence.

Homologous recombination is the ability of complementary DNA sequences to align and exchange regions of homology. For example, transgenic DNA (“donor”) containing sequences homologous to the genomic sequences being targeted (“template”) may be generated and introduced into an organism to undergo recombination with the organism's genomic sequences.

The ability to carry out homologous recombination in a host organism has many practical implications for what can be carried out at the molecular genetic level and is useful in the generation of microbes that produce a desired product. By its very nature, homologous recombination is a precise gene targeting event; hence, most transgenic lines generated with the same targeting sequence will be essentially identical in terms of phenotype, necessitating the screening of far fewer transformation events. Homologous recombination also targets gene insertion events into the host chromosome, potentially resulting in excellent genetic stability, even in the absence of genetic selection.

Because homologous recombination is a precise gene targeting event, it can be used to precisely modify any nucleotide(s) within a gene or region of interest, so long as sufficient flanking regions have been identified. Therefore, homologous recombination can be used to modify the regulatory sequences impacting the expression of RNA and/or proteins. It can also modify protein coding regions, for example, by modifying enzyme activities such as substrate specificity, binding affinities and Km, and thus, it may affect a desired change in the metabolism of a host cell. Homologous recombination provides a powerful means to manipulate the host genome resulting in gene targeting, gene conversion, gene deletion, gene duplication, gene inversion and exchanging gene expression regulatory elements such as promoters, enhancers and 3′UTRs. Thus, homologous recombination allows for the substitution of an endogenous promoter in an organism with a different promoter. An exogenous promoter may provide advantages over the endogenous promoter; for example, the exogenous promoter may increase or decrease the transcription of an operably-linked gene, or the exogenous promoter may allow for the regulation of transcription by different cellular processes relative to the endogenous promoter.

Homologous recombination can be achieved by using targeting constructs containing pieces of endogenous sequences to “target” the gene or region of interest within the endogenous host cell genome. Such targeting sequences can be located upstream or downstream of the gene or region of interest, or flank the gene/region of interest. Such targeting constructs can be transformed into the host cell as circular plasmid DNA, optionally including nucleotide sequences from the plasmid; linearized DNA, such as a plasmid restriction digest; PCR product, such as the amplification of overlapping oligonucleotides; or any other means of introducing DNA into a cell. In some cases, it may be advantageous to first expose the homologous sequences within the transgenic DNA (donor DNA) by cutting the transgenic DNA with a restriction enzyme, which can increase recombination efficiency and decrease the occurrence of non-specific recombination events. Other methods of increasing recombination efficiency include using PCR to generate transforming transgenic DNA containing linear ends homologous to the genomic sequences being targeted.

### E. Transformation

Cells can be transformed by any suitable technique including, e.g., biolistics, electroporation, glass bead transformation, and silicon carbide whisker transformation. Any convenient technique for introducing a transgene into a microorganism can be employed in the present invention. Transformation can be achieved by, for example, the method of D. M. Morrison (Methods in Enzymology 68:326 (1979)), the method by increasing permeability of recipient cells for DNA with calcium chloride (Mandel & Higa, J. Molecular Biology, 53:159 (1970)), or the like.

Examples of the expression of transgenes in oleaginous yeast (e.g., Yarrowia lipolytica) can be found in the literature (Bordes et al., J. Microbiological Methods, 70:493 (2007); Chen et al., Applied Microbiology & Biotechnology 48:232 (1997)).

Vectors for the transformation of microorganisms can be prepared by known techniques. In one embodiment, an exemplary vector for the expression of a gene in a microorganism comprises a gene encoding a protein in operable linkage with a promoter. Alternatively, if the promoter is not operably linked with the gene of interest, the promoter may be transformed into a cell such that it becomes operably linked to a native gene at the point of vector integration. Additionally, microbes may be transformed with two vectors simultaneously (See, e.g., Protist 155:381-93 (2004)). The transformed cells can be optionally selected based upon their ability to grow in the presence of an antibiotic or other selectable marker under conditions in which untransformed cells would not grow.

### Exemplary Nucleic Acids Cells and Methods

1. Nucleotide Sequences Derived from Arxula adeninivorans and Yarrowia lipolytica

In some embodiments, the invention relates to a nucleic acid molecule encoding a promoter. In some embodiments, the promoter is derived from a gene encoding a Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pi cotransporter; Pyruvate decarboxylase; Phytase; or Alpha-amylase. In some embodiments, the promoter is derived from a gene encoding TEF1; GPD1; TPI1; FBA1; GPM1; PYK1; EXP1; RPS7; ADH1; PGK1; HXT7; GAP1; XPR2; ICL1; PDX; MET3; HXK1; SER3; PDA1; PDB1; ACO1; ENO1; ACT1; MDR1; UBI4; YPT1; PHO89; PDC1; PHY; or AMYA.

In some embodiments, the promoter is derived from a gene encoding a Phosphoglycerate kinase; Hexokinase; 6-phosphofructokinase subunit alpha; Triosephosphate isomerase 1; 3-phosphoglycerate dehydrogenase; Pyruvate kinase 1; Pyruvate Dehydrogenase Alpha subunit; Pyruvate Dehydrogenase Beta subunit; Aconitase; Enolase; Actin; Nuclear actin-related protein; Multidrug resistance protein (ABC-transporter); Ubiquitin; Hydrophilic protein involved in ER/Golgi vesicle trafficking; or Plasma membrane Na+/Pi cotransporter. In some embodiments, the promoter is derived from a gene encoding PGK1; HXK1; PFK1; TPI1; SER3; PYK1; PDA1; PDB1; ACO1; ENO1; ACT1; ARP4; MDR1; UBI4; SLY1; or PHO89.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with the sequence set forth in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In other embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with a subsequence of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In some embodiments, the nucleic acid comprises the nucleotide sequence set forth in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In other embodiments, the nucleic acid comprises a nucleotide sequence consisting of a subsequence of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In certain embodiments, the subsequence retains promoter activity. In certain embodiments, the subsequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the subsequence retains the promoter activity of the full-length nucleotide sequence.

In some embodiments, the subsequence is 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 nucleotides long or longer. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In some embodiments, the nucleic acid comprises a nucleotide sequence consisting of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In some embodiments, the nucleic acid comprises a nucleotide sequence consisting of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

2. Vectors Comprising Promoters Derived from Arxula adeninivorans

In some embodiments, the invention relates to a vector comprising a nucleotide sequence encoding a promoter from Arxula adeninivorans, wherein the promoter is derived from a gene encoding a Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pi cotransporter; Pyruvate decarboxylase; Phytase; or Alpha-amylase.

In some embodiments, the vector is a plasmid. In other embodiments, the vector is a linear DNA molecule.

In some embodiments, the vector comprises a nucleotide sequence encoding a promoter from Arxula adeninivorans, wherein the promoter is derived from a gene encoding TEF1; GPD1; TPI1; FBA1; GPM1; PYK1; EXP1; RPS7; ADH1; PGK1; HXT7; GAP1; XPR2; ICL1; PDX; MET3; HXK1; SER3; PDA1; PDB1; ACO1; ENO1; ACT1; ARP4; MDR1; UBI4; YPT1; PHO89; PDC1; PHY; or AMYA.

In some embodiments, the nucleotide sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with the sequence set forth in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In other embodiments, the nucleotide sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with a subsequence of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In some embodiments, the nucleotide sequence comprises the sequence set forth in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In other embodiments, the nucleotide sequence comprises a subsequence of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In certain embodiments, the subsequence retains promoter activity. In other embodiments, the subsequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the subsequence retains the promoter activity of the full-length nucleotide sequence.

In some embodiments, the subsequence is 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 nucleotides long or longer. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53.

In some embodiments, the nucleotide sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In some embodiments, the nucleotide sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

In some embodiments, the nucleotide sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In some embodiments, the nucleotide sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

In some embodiments, the vector further comprises a gene, and the gene and the promoter are operably linked. In other embodiments, the vector is designed so that the promoter becomes operably linked to a gene upon transformation of a cell with the vector.

3. Vectors Comprising Promoters Derived from Yarrowia lipolytica

In some embodiments, the invention relates to a vector comprising a nucleotide sequence encoding a promoter from Yarrowia lipolytica, wherein the promoter is derived from a gene encoding a Phosphoglycerate kinase; Hexokinase; 6-phosphofructokinase subunit alpha; Triosephosphate isomerase 1; 3-phosphoglycerate dehydrogenase; Pyruvate kinase 1; Pyruvate Dehydrogenase Alpha subunit; Pyruvate Dehydrogenase Beta subunit; Aconitase; Enolase; Actin; Nuclear actin-related protein; Multidrug resistance protein (ABC-transporter); Ubiquitin; Hydrophilic protein involved in ER/Golgi vesicle trafficking; or Plasma membrane Na+/Pi cotransporter.

In some embodiments, the vector is a plasmid. In other embodiments, the vector is a linear DNA molecule.

In some embodiments, the vector comprises a nucleotide sequence encoding a promoter from Yarrowia lipolytica, wherein the promoter is derived from a gene encoding PGK1; HXK1; PFK1; TPI1; SER3; PYK1; PDA1; PDB1; ACO1; ENO1; ACT1; ARP4; MDR1; UBI4; SLY1; or PHO89.

In some embodiments, the nucleotide sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with the sequence set forth in SEQ ID NO: 16, 17, 18, 19, 20, 21, 22, 23, 24, 25, 26, 27, 28, 29, 30, 31, 32, 33, or 34. In other embodiments, the nucleotide sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with a subsequence of SEQ ID NO: 16, 17, 18, 19, 20, 21, 22, 23, 24, 25, 26, 27, 28, 29, 30, 31, 32, 33, or 34. In some embodiments, the nucleotide sequence comprises the sequence set forth in SEQ ID NO: 16, 17, 18, 19, 20, 21, 22, 23, 24, 25, 26, 27, 28, 29, 30, 31, 32, 33, or 34. In other embodiments, the nucleotide sequence comprises a subsequence of SEQ ID NO: 16, 17, 18, 19, 20, 21, 22, 23, 24, 25, 26, 27, 28, 29, 30, 31, 32, 33, or 34. In certain embodiments, the subsequence retains promoter activity. In certain embodiments, the subsequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the subsequence retains the promoter activity of the full-length nucleotide sequence.

In some embodiments, the subsequence is 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 nucleotides long or longer. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 16, 17, 18, 19, 20, 21, 22, 23, 24, 25, 26, 27, 28, 29, 30, 31, 32, 33, or 34. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 16, 17, 18, 19, 20, 21, 22, 23, 24, 25, 26, 27, 28, 29, 30, 31, 32, 33, or 34.

In some embodiments, the nucleotide sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 16, 17, 18, 19, 20, 21, 22, 23, 24, 25, 26, 27, 28, 29, 30, 31, 32, 33, or 34. In some embodiments, the nucleotide sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 16, 17, 18, 19, 20, 21, 22, 23, 24, 25, 26, 27, 28, 29, 30, 31, 32, 33, or 34. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

In some embodiments, the nucleotide sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 16, 17, 18, 19, 20, 21, 22, 23, 24, 25, 26, 27, 28, 29, 30, 31, 32, 33, or 34. In some embodiments, the nucleotide sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 16, 17, 18, 19, 20, 21, 22, 23, 24, 25, 26, 27, 28, 29, 30, 31, 32, 33, or 34. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

4. Transformed Cells Comprising Promoters Derived from Arxula adeninivorans, and Methods of Transforming Cells with Promoters Derived from Arxula adeninivorans

In certain aspects, the invention relates to a transformed cell comprising a genetic modification, wherein the genetic modification is transformation with a nucleic acid encoding a promoter from Arxula adeninivorans. In some aspects, the invention relates to methods of expressing a gene in a cell comprising transforming a parent cell with a nucleic acid encoding a promoter from Arxula adeninivorans. In some embodiments, the nucleic acid comprises a gene, and the gene and the promoter are operably linked. In other embodiments, the nucleic acid is designed so that the promoter becomes operably linked to a gene after transformation of the parent cell.

In some embodiments, the promoter is derived from a gene encoding a Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pi cotransporter; Pyruvate decarboxylase; Phytase; or Alpha-amylase. In some embodiments, the promoter is derived from a gene encoding TEF1; GPD1; TPI1; FBA1; GPM1; PYK1; EXP1; RPS7; ADH1; PGK1; HXT7; GAP1; XPR2; ICL1; PDX; MET3; HXK1; SER3; PDA1; PDB1; ACO1; ENO1; ACT1; MDR1; UBI4; YPT1; PHO89; PDC1; PHY; or AMYA.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with the sequence set forth in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In other embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with a subsequence of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In some embodiments, the nucleic acid comprises the nucleotide sequence set forth in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In other embodiments, the nucleic acid comprises a nucleotide sequence consisting of a subsequence of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In certain embodiments, the subsequence retains promoter activity. In certain embodiments, the subsequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the subsequence retains the promoter activity of the full-length nucleotide sequence.

In some embodiments, the subsequence is 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 nucleotides long or longer. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In some embodiments, the nucleic acid comprises a nucleotide sequence consisting of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In some embodiments, the nucleic acid comprises a nucleotide sequence consisting of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 5, 6, 7, 8, 9, 10, 11, 12, 13, 14, 15, 35, 36, 37, 38, 39, 40, 41, 42, 43, 44, 45, 46, 47, 48, 49, 50, 51, 52, or 53. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

5. Transformed Cells Comprising Promoters Derived from Yarrowia lipolytica, and Methods of Transforming Cells with Promoters Derived from Yarrowia lipolytica

In certain aspects, the invention relates to a transformed cell comprising a genetic modification, wherein the genetic modification is transformation with a nucleic acid encoding a promoter from Yarrowia lipolytica. In some aspects, the invention relates to methods of expressing a gene in a cell comprising transforming a parent cell with a nucleic acid encoding a promoter from Yarrowia lipolytica. In some embodiments, the nucleic acid comprises a gene, and the gene and the promoter are operably linked. In other embodiments, the nucleic acid is designed so that the promoter becomes operably linked to a gene after transformation of the parent cell.

In some embodiments, the promoter is derived from a gene encoding a Phosphoglycerate kinase; Hexokinase; 6-phosphofructokinase subunit alpha; Triosephosphate isomerase 1; 3-phosphoglycerate dehydrogenase; Pyruvate kinase 1; Pyruvate Dehydrogenase Alpha subunit; Pyruvate Dehydrogenase Beta subunit; Aconitase; Enolase; Actin; Nuclear actin-related protein; Multidrug resistance protein (ABC-transporter); Ubiquitin; Hydrophilic protein involved in ER/Golgi vesicle trafficking; or Plasma membrane Na+/Pi cotransporter. In some embodiments, the promoter is derived from a gene encoding PGK1; HXK1; PFK1; TPI1; SER3; PYK1; PDA1; PDB1; ACO1; ENO1; ACT1; ARP4; MDR1; UBI4; SLY1; or PHO89.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with the sequence set forth in SEQ ID NO: 16, 17, 18, 19, 20, 21, 22, 23, 24, 25, 26, 27, 28, 29, 30, 31, 32, 33, or 34. In other embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with a subsequence of SEQ ID NO: 16, 17, 18, 19, 20, 21, 22, 23, 24, 25, 26, 27, 28, 29, 30, 31, 32, 33, or 34. In some embodiments, the nucleic acid comprises the nucleotide sequence set forth in SEQ ID NO: 16, 17, 18, 19, 20, 21, 22, 23, 24, 25, 26, 27, 28, 29, 30, 31, 32, 33, or 34. In other embodiments, the nucleic acid comprises a nucleotide sequence consisting of a subsequence of SEQ ID NO: 16, 17, 18, 19, 20, 21, 22, 23, 24, 25, 26, 27, 28, 29, 30, 31, 32, 33, or 34. In certain embodiments, the subsequence retains promoter activity. In certain embodiments, the subsequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the subsequence retains the promoter activity of the full-length nucleotide sequence.

In some embodiments, the subsequence is 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 nucleotides long or longer. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 16, 17, 18, 19, 20, 21, 22, 23, 24, 25, 26, 27, 28, 29, 30, 31, 32, 33, or 34. In some embodiments, the subsequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 16, 17, 18, 19, 20, 21, 22, 23, 24, 25, 26, 27, 28, 29, 30, 31, 32, 33, or 34.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 16, 17, 18, 19, 20, 21, 22, 23, 24, 25, 26, 27, 28, 29, 30, 31, 32, 33, or 34. In some embodiments, the nucleic acid comprises a nucleotide sequence consisting of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides found anywhere in SEQ ID NO: 16, 17, 18, 19, 20, 21, 22, 23, 24, 25, 26, 27, 28, 29, 30, 31, 32, 33, or 34. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

In some embodiments, the nucleic acid comprises a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence homology with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 16, 17, 18, 19, 20, 21, 22, 23, 24, 25, 26, 27, 28, 29, 30, 31, 32, 33, or 34. In some embodiments, the nucleic acid comprises a nucleotide sequence consisting of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 consecutive nucleotides at the 3′-terminus of SEQ ID NO: 16, 17, 18, 19, 20, 21, 22, 23, 24, 25, 26, 27, 28, 29, 30, 31, 32, 33, or 34. In certain embodiments, the nucleotide sequence retains promoter activity. In certain embodiments, the nucleotide sequence retains at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the promoter activity of the full-length nucleotide sequence. In certain embodiments, the nucleotide sequence retains the promoter activity of the full-length nucleotide sequence.

### 6. Species of Cells, Parent Cells, and Transformed Cells

The cell may be selected from the group consisting of algae, bacteria, molds, fungi, plants, and yeasts. In some embodiments, the cell is selected from the group consisting of Arxula, Aspergillus, Aurantiochytrium, Candida, Claviceps, Cryptococcus, Cunninghamella, Geotrichum, Hansenula, Kluyveromyces, Kodamaea, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces, and Yarrowia. In certain embodiments, the cell is selected from the group consisting of Arxula adeninivorans, Aspergillus niger, Aspergillus orzyae, Aspergillus terreus, Aurantiochytrium limacinum, Candida utilis, Claviceps purpurea, Cryptococcus albidus, Cryptococcus curvatus, Cryptococcus ramirezgomezianus, Cryptococcus terreus, Cryptococcus wieringae, Cunninghamella echinulata, Cunninghamella japonica, Geotrichum fermentans, Hansenula polymorpha, Kluyveromyces lactis, Kluyveromyces marxianus, Kodamaea ohmeri, Leucosporidiella creatinivora, Lipomyces lipofer, Lipomyces starkeyi, Lipomyces tetrasporus, Mortierella isabellina, Mortierella alpina, Ogataea polymorpha, Pichia ciferrii, Pichia guilliermondii, Pichia pastoris, Pichia stipites, Prototheca zopfii, Rhizopus arrhizus, Rhodosporidium babjevae, Rhodosporidium toruloides, Rhodosporidium paludigenum, Rhodotorula glutinis, Rhodotorula mucilaginosa, Saccharomyces cerevisiae, Schizosaccharomyces pombe, Tremella enchepala, Trichosporon cutaneum, Trichosporon fermentans, Wickerhamomyces ciferrii, and Yarrowia lipolytica. Thus, the cell may be Yarrowia lipolytica. The cell may be Arxula adeninivorans.

The present description is further illustrated by the following examples, which should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### EXEMPLIFICATION

### Example 1: Sequencing the Arxula adeninivorans Genome and Identifying Promoter Sequences

Arxula adeninivorans promoters were identified and screened. First, in order to access the promoter sequences of selected genes, the genome of A. adeninivorans strain NS252 (ATCC 76597) was sequenced and annotated by Synthetic Genomics Inc. (CA, USA).

Promoters that may be especially useful at driving transcription were enumerated based on published data about commonly used promoters in yeast and fungi. For example, the promoters of genes that are involved in important metabolic pathways such as glycolysis were identified and screened. The A. adeninivorans promoter sequences that may be especially useful at driving transcription are shown in SEQ ID NOs: 5-15 and 35-53 and listed in Table I below.

TABLE I
Arxula adeninivorans promoters

| Promot- | Promoter | | SEQ |
|---|---|---|---|
| er | ID | Associated Protein Function | ID NO |
| TEF1 | PR14 | Translation Elongation factor EF-1α | 5 |
| GPD1 | PR15 | Glycerol-3-phosphate dehydrogenase | 6 |
| TPI1 | PR16 | Triosephosphate isomerase 1 | 7 |
| FBA1 | PR17 | Fructose-1,6-bisphosphate aldolase | 8 |
| GPM1 | PR18 | Phosphoglycerate mutase | 9 |
| PYK1 | PR19 | Pyruvate kinase | 10 |
| EXP1 | PR20 | Export protein | 11 |
| RPS7 | PR21 | Ribosomal protein S7 | 12 |
| ADH1 | PR25 | Alcohol dehydrogenase | 13 |
| PGK1 | PR26 | Phosphoglycerate kinase | 14 |
| HXT7 | PR27 | Hexose Transporter | 15 |
| GAP1 | PR57 | General amino acid permease | 35 |
| XPR2 | PR58 | Serine protease | 36 |
| ICL1 | PR59 | Isocitrate lyase | 37 |
| POX | PR60 | Acyl-CoA oxidase | 38 |
| MET3 | PR61 | ATP-sulfurylase | 39 |
| HXK1 | PR62 | Hexokinase | 40 |
| SER3 | PR63 | 3-phosphoglycerate dehydrogenase | 41 |
| PDA1 | PR64 | Pyruvate Dehydrogenase Alpha subunit | 42 |
| PDB1 | PR65 | Pyruvate Dehydrogenase Beta subunit | 43 |
| ACO1 | PR66 | Aconitase | 44 |
| ENO1 | PR67 | Enolase | 45 |
| ACT1 | PR68 | Actin | 46 |
| MDR1 | PR69 | Multidrug resistance protein (ABC- | 47 |
| | | transporter) | |
| UBI4 | PR70 | Ubiquitin | 48 |
| YPT1 | PR71 | GTPase | 49 |
| PHO89 | PR72 | Plasma membrane Na+/Pi cotransporter | 50 |
| PDC1 | PR73 | Pyruvate decarboxylase | 51 |
| PHY | PR74 | Phytase | 52 |
| AMYA | PR75 | Alpha-amylase | 53 |

### Example 2: Identification of Yarrowia lipolytica Promoters

The Yarrowia lipolytica genome is publically available in the KEGG database, but the precise sequences of each Y. lipolytica promoter have yet to be identified or validated.

Promoters that may be especially useful at driving transcription were enumerated based on published data about commonly used promoters in yeast and fungi. For example, the promoters of genes that are involved in important metabolic pathways such as glycolysis were identified and screened. The Y. lipolytica promoter sequences that may be especially useful at driving transcription are shown in SEQ ID NOs: 16-34 and listed in Table II below.

TABLE II
Yarrowia lipolytica promoters

| Promo- | Promoter | | SEQ |
|---|---|---|---|
| ter | ID | Associated Protein Function | ID NO |
| PGK1 | PR34*, PR54 | Phosphoglycerate kinase | 16*, 32 |
| HXK1 | PR35 | Hexokinase | 17 |
| PFK1 | PR36 | 6-phosphofructokinase subunit alpha | 18 |
| TPI1 | PR37*, PR55 | Triosephosphate isomerase 1 | 19*, 33 |
| SER3 | PR38 | 3-phosphoglycerate dehydrogenase | 20 |
| PYK1 | PR39*, PR56 | Pyruvate kinase 1 | 21*, 34 |
| PDA1 | PR40 | Pyruvate Dehydrogenase Alpha | 22 |
| | | subunit | |
| PDB1 | PR41 | Pyruvate Dehydrogenase Beta subunit | 23 |
| ACO1 | PR42 | Aconitase | 24 |
| ENO1 | PR43 | Enolase | 25 |
| ACT1 | PR44 | Actin | 26 |
| ARP4 | PR45 | Nuclear actin-related protein | 27 |
| MDR1 | PR46 | Multidrug resistance protein (ABC- | 28 |
| | | transporter) | |
| UBI4 | PR47 | Ubiquitin | 29 |
| SLY1 | PR49 | Hydrophilic protein involved in | 30 |
| | | ER/Golgi vesicle trafficking | |
| PHO89 | PR50 | Plasma membrane Na+/Pi | 31 |
| | | cotransporter | |

*Denotes promoter and contiguous transcribed sequence.

### Example 3: Validating Yarrowia lipolytica Promoter Sequences and Assessing their Strength Using an Invertase Reporter Gene

Selected Yarrowia lipolytica promoters were screened in Y. lipolytica strain NS18 for functionality and strength using the Saccharomyces cerevisiae invertase gene SUC2 (SEQ ID NO:1) as a reporter. The invertase gene was used as both a selection marker, for screening cells for growth on sucrose, and as a reporter for the quantitative evaluation of a promoter's strength. Additionally, promoter strengths were measured by the DNS assay described in Example 4.

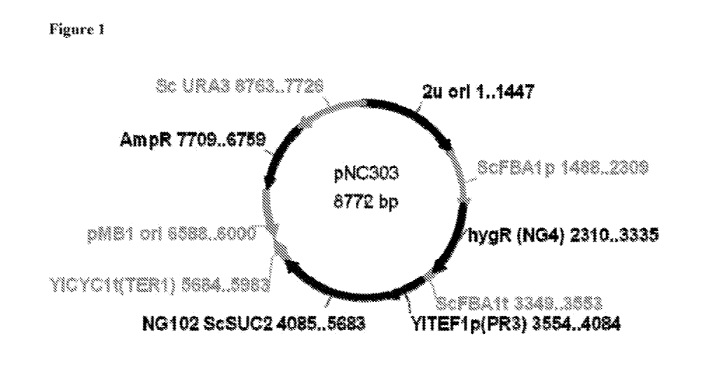

The S. cerevisiae invertase gene was expressed in Y. lipolytica strain NS18 under the control of fourteen different Y. lipolytica promoters and the same TER1 terminator. Promoters were amplified from the genomic DNA of host Y. lipolytica strain NS18 (obtained from NRRL # YB-392) using reverse primers that contained 30-35 base pairs homologous with the 5′ end of the invertase gene to allow for homologous recombination of the promoter and invertase DNA. The invertase nucleotide sequence and TER1 terminator were amplified from the pNC303 plasmid (FIG. 1). DNA for each amplified promoter was combined with the DNA for the amplified invertase-TER1 fragment and transformed into the NS18 strain using the transformation protocol described in Chen et al. (Applied Microbiology & Biotechnology 48:232-35 (1997)). The promoter DNA fragments and the invertase-TER1 DNA fragments assembled in vivo and randomly integrated into the genome of the host Y. lipolytica strain NS18.

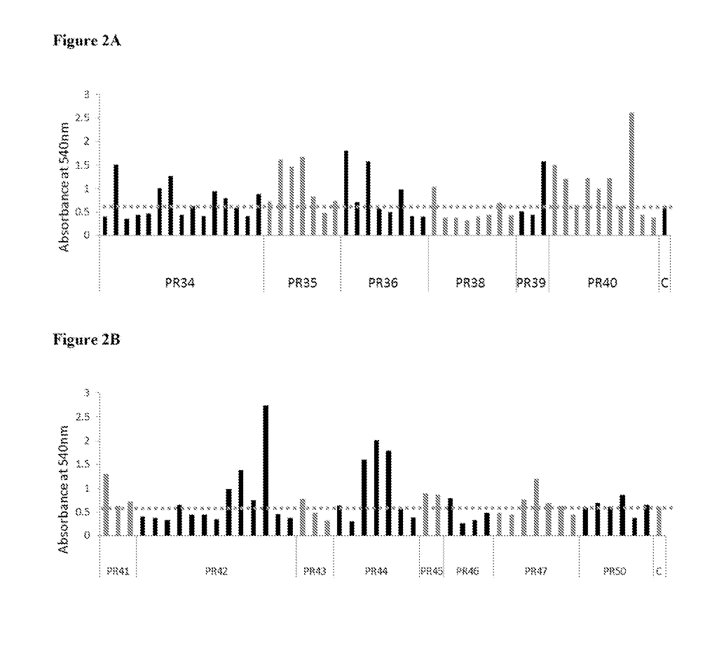

Transformants were plated and selected on YNB plates with 2% sucrose and screened for invertase activity by the DNS assay described in Example 4. Several transformants were analysed for each promoter. The results of the DNS assay are shown in the FIG. 2. Most promoters displayed significant colony variation between the transformants, possibly due to the effect of the invertase's site of integration on expression. FIG. 2 demonstrates that all fourteen promoters allow for invertase expression. For those promoters with lower expression levels and lower colony numbers (PR39, PR41, PR43, PR45, and PR46), the fact that their transfomants grew on YNB+2% sucrose selective plates demonstrates that the promoters nevertheless enabled sufficient transcription of invertase to allow for growth on sucrose.

### Example 4: Dinitrosalicylic Acid Assay

Cells were incubated at 30° C. on YPD agar plates for one to two days. Cells from each agar plate were used to inoculate 300 μL of media in the wells of a 96-well plate. The 96-well plates were covered with a porous cover and incubated at 30° C., 70-90% humidity, and 900 rpm in an Infors Multitron ATR shaker.

The 96-well plates were centrifuged at 3000 rpm for 2 minutes. 50 μL of the supernatant was added to 150 μL of 50 mM sucrose containing 40 mM sodium acetate, pH 4.5-5, in a new 96-well plate and incubated at 30° C. for 30-60 minutes.

30 μL of the sucrose/supernatant mixture was added to 60 μL of DNS reagent (1% dinitrosalicylic acid, 30% sodium potassium tartrate, 0.4 M NaOH) in a fresh 96-well plate and covered with PCR film. The plate was heated to 99° C. in a thermocycler for 5 minutes. 70 μL of the mixture was then transferred into a Corning 96-well clear flat bottom plate, and the absorbance at 540 nm was monitored on a SpectraMax M2 spectrophotometer (Molecular Devices).

### Example 5: Validating Arxula adeninivorans Promoter Sequences Using a hygR Reporter Gene

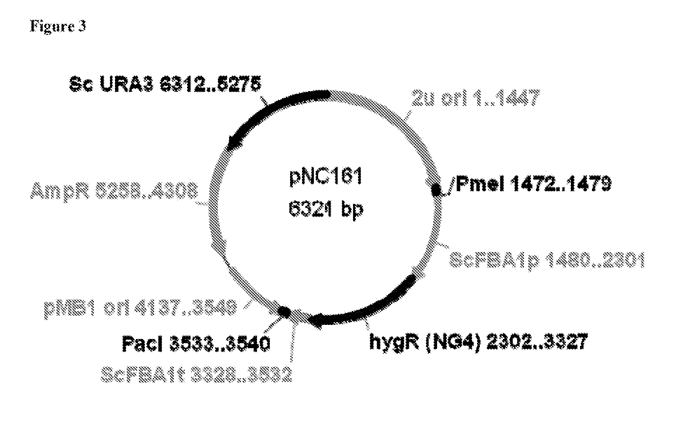

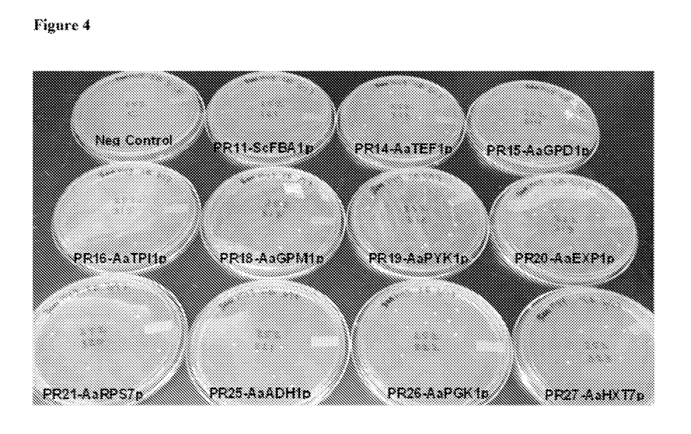

The invertase reporter assays described in Examples 3 and 4 were not amenable to A. adeninivorans strain NS252 because this strain has the native ability to grow on sucrose. Therefore, the Escherichia coli hygR gene (SEQ ID NO:2) was used as a reporter in A. adeninivorans and as a transformation selection marker for selection with Hygromycin B (HYG). The hygR gene was expressed in Y. lipolytica and A. adeninivorans under the control of eleven selected promoters and the same terminator (FIGS. 4 & 5). FIG. 3 shows a map of the expression construct pNC161 used to overexpress the hygR gene in Y. lipolytica and A. adeninivorans using the FBA1 promoter from S. cerevisiae (SEQ ID NO:4) as an example. The FBA1 promoter was also used as a positive control because it can drive hygR expression in both Y. lipolytica and A. adeninivorans. All hygR expression constructs were identical to pNC161 except for the promoter sequences. Cells were transformed with water as a negative control.

The expression constructs were linearized prior to transformation by a PacI/PmeI restriction digest. Each linear expression construct included the expression cassette for the hygR gene and a different promoter. The expression constructs were randomly integrated into the genome of Y. lipolytica strain NS18 and A. adeninivorans strain NS252 using the transformation protocol described in Chen et al. (Applied Microbiology & Biotechnology 48:232-35 (1997)).

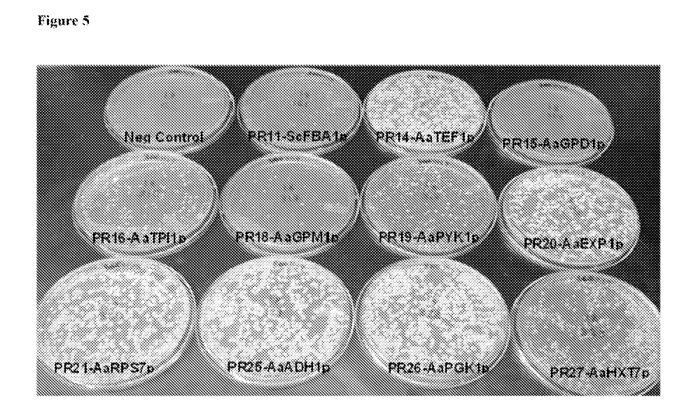

The transformants were selected on YPD plates with 300 μg/mL HYG and screened for promoter strength based on the size of the colonies that grew on the plates. Pictures of the YPD+HYG plates with each transformant are shown in FIGS. 4 & 5. The transformation efficiency for A. adeninivorans was much lower than Y. lipolytica, likely because the transformation protocol was optimized for Y. lipolytica rather than A. adeninivorans. The number of transformants varied between the different constructs, likely due to a slightly different amount of DNA used during different transformations, although promoter strength may have contributed to this variation. FIGS. 4 and 5 nevertheless demonstrate that all eleven promoters are functional in both Y. lipolytica and A. adeninivorans.

The size of colonies for the A. adeninivorans transformants did not vary significantly for different A. adeninivorans promoters, indicating that the native A. adeninivorans promoters had similar efficiency when linked to the hygR reporter. At the same time, the size of the Y. lipolytica colonies varied significantly. This data may suggest that different A. adeninivorans promoters interact similarly with A. adeninivorans regulating factors and differently with Y. lipolytica regulating factors.

Every promoter screened in both Arxula adeninivorans and Yarrowia lipolytica was capable of driving gene expression in both Arxula adeninivorans and Yarrowia lipolytica, which suggests that all of the promoters identified in SEQ ID NOs:6-53 are functional in all yeast.

### Example 6: Assessing the Strength of Arxula adeninivorans and Yarrowia lipolytica Promoter Sequences Using DGA2 as a Reporter

The most efficient promoters as assessed by the invertase and hygR assays described in Examples 3-5 were selected for further quantitative testing in Y. lipolytica using the diacylglycerol acyltransferase DGA1 as a reporter. The DGA1 protein catalyses the final step of the synthesis of triacylglycerol (TAG), and thus, DGA1 is a key component in the lipid synthesis pathway. DGA1 overexpression in Y. lipolytica significantly increases its lipid production efficiency. Therefore, a promoter's strength in the DGA1 assay correlates with lipid production efficiency.

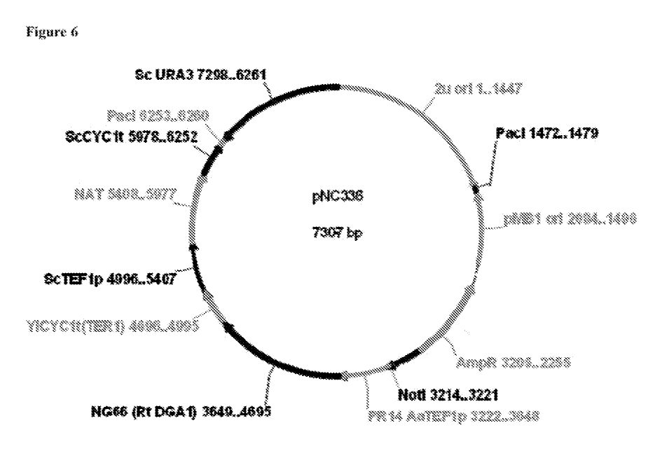

The gene encoding DGA1 from Rhodosporidium toruloides (SEQ ID NO:3) was expressed in Y. lipolytica under the control of twelve selected promoters and the same terminator. FIG. 6 shows a map of the expression construct pNC336 as example; this construct was used to overexpress DGA1 with the TEF1 promoter from A. adeninivorans (SEQ ID NO:5). All other DGA1 expression constructs were identical to pNC336 except for their promoter sequences.

The expression constructs were linearized prior to transformation by PacI/NotI restriction digest. Each linear expression construct included the expression cassette for the gene encoding DGA1 and for the Nat1 gene used as a marker for selection with nourseothricin (NAT). The expression constructs were randomly integrated into the genome of Y. lipolytica strain NS18 using the transformation protocol described in Chen et al. (Applied Microbiology & Biotechnology 48:232-35 (1997)). Transformants were selected on YPD plates with 500 μg/mL NAT and screened for ability to accumulate lipids by the fluorescent staining lipid assay described in Example 7.

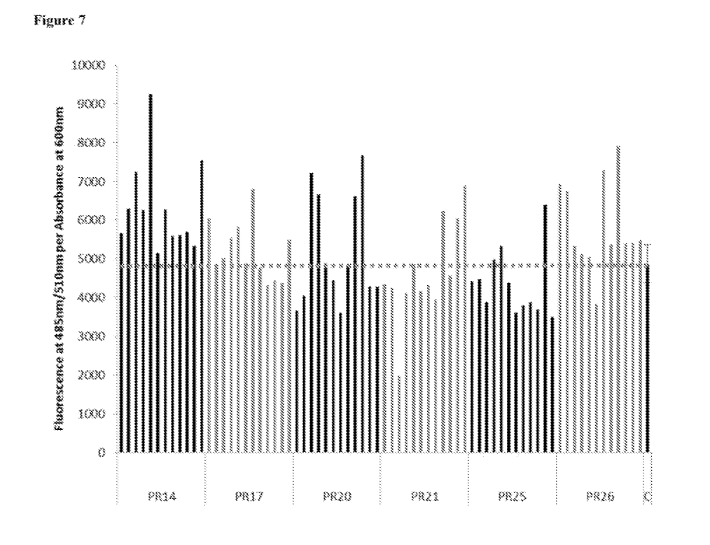

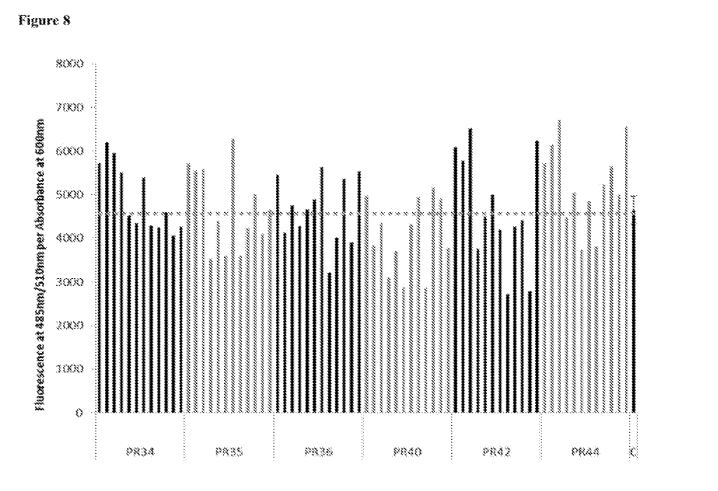

Twelve transformants were analysed for each expression construct using the fluorescent staining lipid assay described in Example 7 (FIGS. 7 & 8). Most constructs displayed significant colony variation between transformants, possibly due to either the lack of a functional DGA1 expression cassette in some transformants that only obtained a functional Nat1 cassette or the negative effect of the DGA1 expression cassette site of integration on DGA1 expression. Nevertheless, FIGS. 7 and 8 demonstrate that all twelve promoters increased the lipid content of Y. lipolytica, which confirms the functionality of each promoter for increasing lipid production and reconfirms their functionality for driving gene expression.

### Example 7: Lipid Fluorescence Assay

Each well of an autoclaved, multi-well plate was filled with filter-sterilized media containing 0.5 g/L urea, 1.5 g/L yeast extract, 0.85 g/L casamino acids, 1.7 g/L YNB (without amino acids and ammonium sulfate), 100 g/L glucose, and 5.11 g/L potassium hydrogen phthalate (25 mM). 1.5 mL of media was used per well for 24-well plates and 300 μl of media was used per well for 96-well plates. Alternatively, the yeast cultures were used to inoculate 50 ml of sterilized media in an autoclaved 250 mL flask. Yeast strains that had been incubated for 1-2 days on YPD-agar plates at 30° C. were used to inoculate each well of the multiwall plate.

Multi-well plates were covered with a porous cover and incubated at 30° C., 70-90% humidity, and 900 rpm in an Infors Multitron ATR shaker. Alternatively, flasks were covered with aluminum foil and incubated at 30° C., 70-90% humidity, and 900 rpm in a New Brunswick Scientific shaker. After 96 hours, 20 μL of 100% ethanol was added to 20 μL of cells in an analytical microplate and incubated at 4° C. for 30 minutes. 20 μL of cell/ethanol mix was then added to 80 μL of a pre-mixed solution containing 50 μL 1 M potassium iodide, 1 mM μL Bodipy 493/503, 0.5 μL 100% DMSO, 1.5 μL 60% PEG 4000, and 27 μL water in a Costar 96-well, black, clear-bottom plate and covered with a transparent seal. Bodipy fluorescence was monitored with a SpectraMax M2 spectrophotometer (Molecular Devices) kinetic assay at 30° C., and normalized by dividing fluorescence by absorbance at 600 nm.

### Example 8: Arxula adeninivorans Promoters to Increase Lipid Production in Yeast

Promoters as assessed by the hygR assays described in Example 5 were selected to screen genes encoding the diacylglycerol acyltransferases (DGAs) from various organisms in Arxula adeninivorans, in order to increase lipid production. The DGA proteins catalyze the final steps of the synthesis of triacylglycerol (TAG), and thus, DGA is a key component in the lipid synthesis pathway.

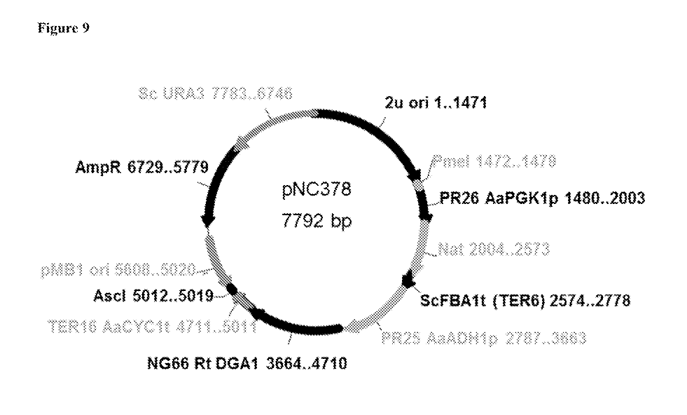

Genes encoding DGA1, DGA2 and DGA3 from various host organisms, such as Arxula adeninivorans, Yarrowia lipolytica, Rhodosporidium toruloides, Lipomyces starkeyi, Aspergillus terreus, Claviceps purpurea, Aurantiochytrium limacinum, Chaetomium globosum, Rhodotorula graminis, Microbotryum violaceum, Puccinia graminis, Gloeophyllum trabeum, Rhodosporidium diobovatum, Phaeodactylum tricornutum, Ophiocordyceps sinensis, Trichoderma virens, Ricinus communis, and Arachis hypogaea, were expressed in A. adeninivorans strain NS252 under the control of the A. adeninivorans ADH1 promoter (SEQ ID NO:13) and CYC1 terminator. FIG. 9 shows a map of the expression construct pNC378 as an example. This construct was used to overexpress Rhodosporidium toruloides DGA1 with the promoter ADH1 from A. adeninivorans (SEQ ID NO: 13). All other DGA expression constructs were identical to pNC378 except for the DGA sequences. The A. adeninivorans PGK1 promoter (SEQ ID NO:14) was used to drive the expression of the selection marker NAT in all constructs.

TABLE III
List of DGAs Screened using the
A. Adeninivorans ADH1 promoter

| Gene | Gene ID | Donor Organism |
|---|---|---|
| DGA2 | NG168 | Arxula adeninivorans |
| DGA1 | NG167 | Arxula adeninivorans |
| DGA1 | NG15 | Yarrowia lipolytica |
| DGA1 | NG66 | Rhodosporidium toruloides |
| DGA1 | NG69 | Lipomyces starkeyi |
| DGA1 | NG70 | Aspergillus terreus |
| DGA1 | NG71 | Claviceps purpurea |
| DGA1 | NG72 | Aurantiochytrium limacinum |
| DGA2 | NG16 | Yarrowia lipolytica |
| DGA2 | NG109 | Rhodosporidium toruloides |
| DGA2 | NG110 | Lipomyces starkeyi |
| DGA2 | NG111 | Aspergillus terreus |
| DGA2 | NG112 | Claviceps purpurea |
| DGA2 | NG113 | Chaetomium globosum |
| DGA1 | NG286 | Rhodotorula graminis |
| DGA1 | NG287 | Microbotryum violaceum |
| DGA1 | NG288 | Puccinia graminis |
| DGA1 | NG289 | Gloeophyllum trabeum |
| DGA1 | NG290 | Rhodosporidium diobovatum |
| DGA1 | NG293 | Phaeodactylum tricornutum |
| DGA2 | NG295 | Phaeodactylum tricornutum |
| DGA2 | NG297 | Ophiocordyceps sinensis |
| DGA2 | NG298 | Trichoderma virens |
| DGA3 | NG299 | Ricinus communis |
| DGA3 | NG300 | Arachis hypogaea |

The expression constructs were linearized prior to transformation with a PmeI/AscI restriction digest. Each linear expression construct included the expression cassette for the gene encoding a DGA and the Nat1 gene used as a marker for selection with nourseothricin (NAT). The expression constructs were randomly integrated into the genome of A. adeninivorans strain NS252. Briefly, 5 mL of YPD media was inoculated with NS252 from an overnight colony on a YPD plate and incubated at 37° C. for 16-24 hours. Next, 2.5 mL of the overnight culture was used to inoculate 22.5 mL of YPD media in a 250 mL shake flask. After 3-4 hours at 37° C., the culture was centrifuged at 3000 rpm for 3 minutes. The supernatant was discarded and the cells were washed with water, centrifuged, and the supernatant was discarded.

In order to make the cells competent, 2 mL of 100 mM LiAc and 40 μL of 2 M DTT was added to the cell pellet and incubated at 37° C. for an hour. The cell solution was centrifuged for 10 seconds at 10,000 rpm and the supernatant was discarded. The pellet was first washed with water and then with cold 1 M sorbitol. The washed pellet was resuspended in 2 mL of cold 1M sorbitol and placed on ice. 40 μL of the cell-sorbitol solution and 5 μL of the digested construct were added into pre-chilled 0.2 cm electroporation cuvettes. The cells were electroporated at 25 μF, 200 ohms and 1.5 kV with a time constant ˜4.9-5.0 ms. The cells were recovered in 1 mL YPD at 37° C. overnight. 100 μL-500 μL of the recovered culture was plated on YPD plates with 50 μg/mL NAT.

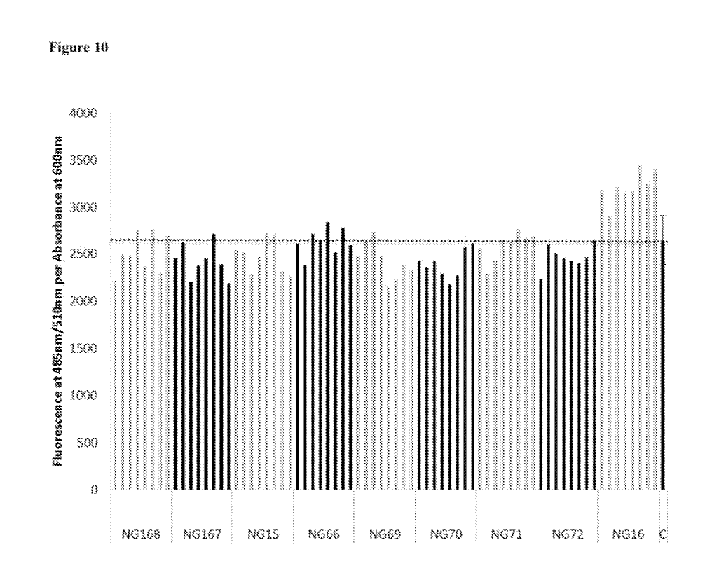

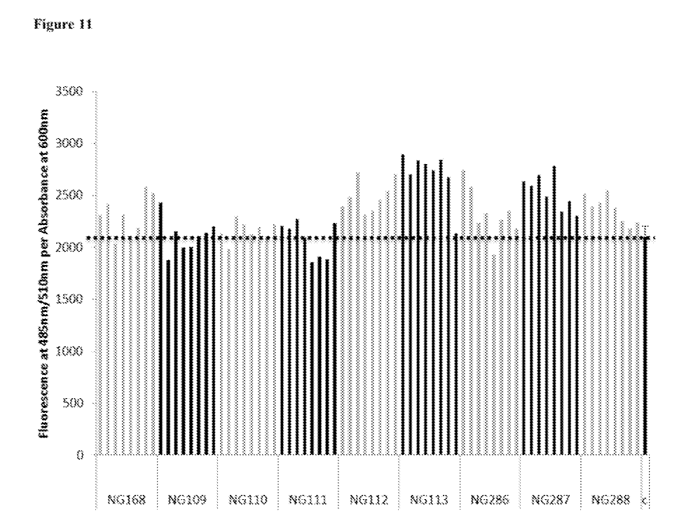

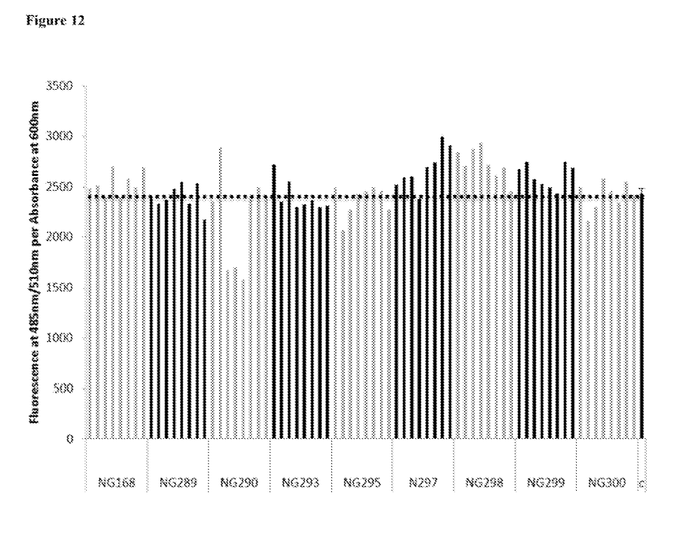

Eight transformants were analysed for each expression construct using the fluorescent staining lipid assay described in Example 7. Most constructs displayed significant colony variation between transformants, possibly due to either the lack of a functional DGA expression cassette in some transformants that only obtained a functional Nat1 cassette or the negative effect of the DGA expression cassette site of integration on DGA expression. Nevertheless, FIGS. 10, 11, and 12 demonstrate that both A. adeninivorans promoters ADH1 and PGK1 are useful as tools to construct viable expression cassettes.

### INCORPORATION BY REFERENCE

All of the patents, published patent applications, and other documents cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A nucleic acid encoding a promoter from Arxula adeninivorans, wherein the promoter is a promoter for Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pi cotransporter; Pyruvate decarboxylase; Phytase; or Alpha-amylase.

2. The nucleic acid of claim 1, wherein the promoter is derived from a gene encoding TEF1; GPD1; TPI1; FBA1; GPM1; PYK1; EXP1; RPS7; ADH1; PGK1; HXT7; GAP1; XPR2; ICU; PDX; MET3; HXK1; SER3; PDA1; PDB1; ACO1; ENO1; ACT1; MDR1; UBI4; YPT1; PHO89; PDC1; PHY; or AMYA.

3. The nucleic acid of claim 1, wherein:
the nucleic acid has at least 90% sequence homology with the nucleotide sequence set forth in SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; or SEQ ID NO:53; or
the nucleic acid has at least 90% sequence homology with a subsequence of SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; or SEQ ID NO:53, and said subsequence retains promoter activity.

4. The nucleic acid of claim 3, wherein the nucleic acid comprises a subsequence of SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; or SEQ ID NO:53, and said subsequence retains promoter activity.

5. The nucleic acid of claim 3, wherein the nucleic acid comprises the nucleotide sequence set forth in SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; or SEQ ID NO:53.

6. The nucleic acid of claim 1, further comprising a gene, wherein the promoter and the gene are operably linked.

7. A vector, comprising a nucleic acid of claim 1.

8. The vector of claim 7, wherein the vector is a plasmid.

9. A transformed cell, comprising the nucleic acid of claim 1.

10. A transformed cell, comprising a genetic modification, wherein said genetic modification is transformation with a nucleic acid encoding a promoter, wherein the promoter has at least 90% sequence homology with a subsequence of SEQ ID NO: 5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; or SEQ ID NO: 53, and said subsequence retains promoter activity.

11. The transformed cell of claim 9, wherein said cell is selected from the group consisting of algae, bacteria, molds, fungi, plants, and yeasts.

12. The transformed cell of claim 11, wherein said cell is a yeast.

13. The transformed cell of claim 12, wherein said cell is selected from the group consisting of Arxula, Aspergillus, Aurantiochytrium, Candida, Claviceps, Cryptococcus, Cunninghamella, Geotrichum, Hansenula, Kluyveromyces, Kodamaea, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces, and Yarrowia.

14. The transformed cell of claim 13, wherein said cell is selected from the group consisting of Aspergillus niger, Aspergillus orzyae, Aspergillus terreus, Aurantiochytrium limacinum, Candida utilis, Claviceps purpurea, Cryptococcus albidus, Cryptococcus curvatus, Cryptococcus ramirezgomezianus, Cryptococcus terreus, Cryptococcus wieringae, Cunninghamella echinulata, Cunninghamella japonica, Geotrichum fermentans, Hansenula polymorpha, Kluyveromyces lactis, Kluyveromyces marxianus, Kodamaea ohmeri, Leucosporidiella creatinivora, Lipomyces lipofer, Lipomyces starkeyi, Lipomyces tetrasporus, Mortierella isabellina, Mortierella alpina, Ogataea polymorpha, Pichia ciferrii, Pichia guilliermondii, Pichia pastoris, Pichia stipites, Prototheca zopfii, Rhizopus arrhizus, Rhodosporidium babjevae, Rhodosporidium toruloides, Rhodosporidium paludigenum, Rhodotorula glutinis, Rhodotorula mucilaginosa, Saccharomyces cerevisiae, Schizosaccharomyces pombe, Tremella enchepala, Trichosporon cutaneum, Trichosporon fermentans, and Wickerhamomyces ciferrii.

15. The transformed cell of claim 13, wherein said cell is Yarrowia lipolytica.

16. The transformed cell of claim 13, wherein said cell is Arxula adeninivorans.

17. A method for expressing a gene in a cell, comprising transforming a parent cell with a nucleic acid encoding a promoter, wherein:
the promoter has at least 90% sequence homology with a subsequence of SEQ ID NO: 5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; or SEQ ID NO: 53;
said subsequence retains promoter activity; and either:
the nucleic acid comprises the gene, and the gene and the promoter are operably linked; or
the nucleic acid is designed so that the promoter becomes operably linked to the gene after transformation of the parent cell.

18. A method for expressing a gene in a cell, comprising transforming a parent cell with a nucleic acid of claim 1;
wherein:
the nucleic acid comprises the gene, and the gene and the promoter are operably linked; or
the nucleic acid is designed so that the promoter becomes operably linked to the gene after transformation of the parent cell.

19. The method of claim 17, wherein the nucleic acid comprises the gene, and the gene and the promoter are operably linked.

20. The method of claim 17, wherein the nucleic acid is designed so that the promoter becomes operably linked to the gene after transformation of the parent cell.

21. The method of claim 17, wherein said cell is a yeast.

22. The method of claim 21, wherein said cell is selected from the group consisting of Arxula, Aspergillus, Aurantiochytrium, Candida, Claviceps, Cryptococcus, Cunninghamella, Geotrichum, Hansenula, Kluyveromyces, Kodamaea, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces, and Yarrowia.

23. The method of claim 22, wherein said cell is selected from the group consisting of Aspergillus niger, Aspergillus orzyae, Aspergillus terreus, Aurantiochytrium limacinum, Candida utilis, Claviceps purpurea, Cryptococcus albidus, Cryptococcus curvatus, Cryptococcus ramirezgomezianus, Cryptococcus terreus, Cryptococcus wieringae, Cunninghamella echinulata, Cunninghamella japonica, Geotrichum fermentans, Hansenula polymorpha, Kluyveromyces lactis, Kluyveromyces marxianus, Kodamaea ohmeri, Leucosporidiella creatinivora, Lipomyces lipofer, Lipomyces starkeyi, Lipomyces tetrasporus, Mortierella isabellina, Mortierella alpina, Ogataea polymorpha, Pichia ciferrii, Pichia guilliermondii, Pichia pastoris, Pichia stipites, Prototheca zopfii, Rhizopus arrhizus, Rhodosporidium babjevae, Rhodosporidium toruloides, Rhodosporidium paludigenum, Rhodotorula glutinis, Rhodotorula mucilaginosa, Saccharomyces cerevisiae, Schizosaccharomyces pombe, Tremella enchepala, Trichosporon cutaneum, Trichosporon fermentans, and Wickerhamomyces ciferrii.

24. The method of claim 22, wherein said cell is Yarrowia lipolytica.

25. The method of claim 22, wherein said cell is Arxula adeninivorans.
